(19) United States
(12) Patent Application Publication
(10) Pub. No.: US 2024/0092898 A1
(43) Pub. Date: Mar. 21, 2024

(54) BISPECIFIC ANTIBODIES BINDING TO CD7 AND CD33

(71) Applicant: BIVICTRIX LIMITED, Macclesfield Cheshire (GB)

(72) Inventor: Tiffany Jane Daniels, Macclesfield Cheshire (GB)

(21) Appl. No.: 18/265,781

(22) PCT Filed: Dec. 23, 2021
(86) PCT No.: PCT/GB2021/053433

§ 371 Date: Jun. 7, 2023

(30) Foreign Application Priority Data
Dec. 23, 2020 (GB) 2020573.8

(51) Int. Cl. C07K 16/28; A61K 39/00; A61K 47/68; A61P 35/02

(52) U.S. Cl. CPC C07K 16/2803; A61K 39/4611; A61K 39/4631; A61K 39/4643; A61K 47/6803; A61K 47/68031; A61K 47/6849; A61P 35/02; C07K 2317/31

(57) ABSTRACT

The present invention relates bispecific antibodies and antigen binding fragments thereof for binding to CD33 and CD7 for use in treating CD33+ CD7+ hematological malignancies, and in particular Acute Myeloid Leukaemia (AML). In particular, the present invention relates to a bispecific antibody or antigen binding fragments thereof binding to CD33 and CD7, wherein the bispecific antibody or antigen binding fragments comprises: a first binding region binding to human CD33 which has a K_{D} binding affinity to CD33 of between about 1.36×10⁹ and about 9.23×10⁻⁸; and a second binding region binding to human CD7 which has a K_{D} binding affinity to CD7 of between about 3.56×10⁻⁹ and about 5.41×10⁻⁷.

### TECHNICAL FIELD OF INVENTION

The present invention relates bispecific antibodies and antigen binding fragments thereof for binding to CD33 and CD7 for use in treating hematological malignancies, and in particular Acute Myeloid Leukaemia (AML).

### BACKGROUND TO THE INVENTION

Acute Myelogenous Leukaemia or Acute Myeloid Leukaemia (AML) is a heterogeneous haematological malignancy involving the clonal expansion of myeloid blasts in the bone marrow and peripheral blood. AML represents >90% of cases of adult acute leukaemia and remains a largely aggressive disease with a fulminant clinical course. Despite advances in therapeutic regimens and the current understanding of successful Haematopoietic Stem Cell Transplantation (HSCT), the mortality rate for patients with AML is still high.

It is well known in the prior art that CD7 and CD33 are both internalising cell surface antigen receptors and that CD7 is a rapid and efficient internalising cell surface antigen receptor (internalisation is seen within 15-30 minutes) whereas CD33 is a slow and inefficient internalising cell surface antigen receptor (internalisation seen after 60 minutes).

WO2019/102234 discloses the dual targeting of cell inhibiting agents to the cell surface receptors CD7 and CD33 in the treatment of haematological malignancy.

Based on the current state of the art and previous experimental work with monovalent CD7 and CD33 binding antibodies, it is expected that the CD33 Fab arm of a bispecific antibody would be sufficient to render a bispecific antibody comprising the CD33 Fab arm cytotoxic to CD33 expressing cells. Additionally, exposing CD7 expressing cells to a CD33+/CD7+ bispecific antibody has been shown to cause substantial cytotoxicity.

It is an object of the present invention to provide improved therapies for haematological malignancies, and in particular AML. It is desirable, if such therapies did not result in myelosuppression.

### SUMMARY OF THE INVENTION

In accordance with a first aspect of the present invention, there is provided a bispecific antibody or antigen binding fragments thereof binding to CD33 and CD7, wherein the bispecific antibody or antigen binding fragments comprises:
a first binding region binding to human CD33 which has a K_{D} binding affinity to CD33 of between about 1.36×10⁻⁹ and about 9.23×10⁻⁸; and
a second binding region binding to human CD7 which has a K_{D} binding affinity to CD7 of between about 3.56×10⁻⁹ and about 2.29×10⁻⁷.

The first binding region binding to human CD33 may have a K_{D} binding affinity to CD33 of about 1.36×10⁻⁹ and the second binding region binding to human CD7 which has a K_{D} binding affinity to CD7 of about 4.52×10⁻⁸.

In certain embodiments, the first binding region will preferably have a reduced K_{D} binding affinity for CD33 compared to a wild type binding region that is able to bind to CD33. The first binding region may have a reduced K_{D} binding affinity for CD33 of up to about 70 fold, or up to about 68 fold. In one embodiment, the first binding region does not has a reduced K_{D} binding affinity for CD33 compared to a wild type binding region.

The certain other embodiments, the second binding region will preferably have a reduced binding affinity for CD7 compared to a wild type binding region that is able to bind to CD7. The second binding region may have a reduced K_{D} binding affinity for CD7 of up to about 90 fold, or up to about 87 fold, or in the range of about 9 to about 90 fold, or in the range of about 9 to about 87 fold.

In an embodiment, both the first and/or second binding regions have a reduced binding affinity for their respective binding targets compared to respective wildtype binding regions ability to bind to the respective binding targets.

The first binding region may have substantially the same K_{D} binding affinity to CD33 when compared to a wild type binding region that is able to bind to CD33 and the second binding region may have a reduced K_{D} binding affinity for CD7 when compared to a wild type binding region that is able to bind to CD7.

The second binding region may have a K_{D} binding affinity for CD7 that is between about 12.70 and about 87 fold lower than that of a wild type binding region that is able to bind to CD7.

The antibody or antigen binding fragments thereof may comprise:
a) the first binding region has a K_{D} binding affinity for CD33 that is selected from one of the following: about 1.36×10⁻⁹ or about 9.23×10⁻⁸; and/or
b) the second binding region binding to human CD7 has a K_{D} binding affinity to CD7 selected from one of the following: about 3.56×10⁻⁹, about 7.57×10⁻⁸, about 7.22×10⁻⁸, about 3.25×10⁻⁸, about 4.52×10⁻⁸, about 5.41×10⁻⁷ or about 2.29×10⁻⁷.

The antibody or antigen binding fragments thereof as described above may have a fold selectivity of CD7⁺/CD33⁺ vs CD7⁺/CD33⁻ in the range of about 2 to about 20 and a fold selectivity of CD7⁺/CD33⁺ vs CD7⁻/CD33⁺ in the range of about 20 to about 70, or a fold selectivity of CD7⁺/CD33⁺ vs CD7⁺/CD33⁻ in the range of about 5 to about 15 and a fold selectivity of CD7⁺/CD33⁺ vs CD7⁻/CD33⁺ in the range of about 20 to about 60, or a fold selectivity of CD7⁺/CD33⁺ vs CD7⁺/CD33⁻ in the range of about 11 to about 15 and a fold selectivity of CD7⁺/CD33⁺ vs CD7⁻/CD33⁺ in the range of about 25 to about 55.

The antibody or antigen binding fragments thereof have a fold selectivity of:
a) CD7⁺/CD33⁺ vs CD7⁺/CD33⁻ of about 14 and a fold selectivity of CD7⁺/CD33⁺ vs CD7⁻/CD33⁺ of about 48;
b) CD7⁺/CD33⁺ vs CD7⁺/CD33⁻ of about 6 and a fold selectivity of CD7⁺/CD33⁺ vs CD7⁻/CD33⁺ of about 48; or
c) CD7⁺/CD33⁺ vs CD7⁺/CD33⁻ of about 7 and a fold selectivity of CD7⁺/CD33⁺ vs CD7⁻/CD33⁺ of about 27.

Preferably, the antibody or antigen binding fragments thereof have a fold selectivity of:
a) CD7⁺/CD33⁺ vs CD7⁺/CD33⁻ of about 13.6 and a fold selectivity of CD7⁺/CD33⁺ vs CD7⁻/CD33⁺ of about 48.3;
b) CD7⁺/CD33⁺ vs CD7⁺/CD33⁻ of about 6.5 and a fold selectivity of CD7⁺/CD33⁺ vs CD7⁻/CD33⁺ of about 27.4; or
c) CD7⁺/CD33⁺ vs CD7⁺/CD33⁻ of about 6.1 and a fold selectivity of CD7⁺/CD33⁺ vs CD7⁻/CD33⁺ of about 47.5.

In an embodiment, there is provided a bispecific antibody or antigen binding fragments thereof binding to CD33 and CD7, wherein the bispecific antibody or antigen binding fragments comprises:
a first binding region binding to human CD33 which does not have a fold reduction in binding affinity relative to wild type CD33; and
a second binding region binding to human CD7 which has a fold reduction in binding affinity relative to wild type CD7 in the region of 12.7.

The antibody or antigen binding fragments thereof as herein above described with reference to all aspects may be for use in the treatment of a CD7+CD33+ malignancy, such as a hematological malignancy.

The antibody or antigen binding fragments thereof as herein above described with reference to all aspects may be for a method of treating a CD7+CD33+ hematological malignancy in an individual in need therefore, where the method comprises administering the antibody or antigen binding fragments thereof. It is preferred that the antibody or antigen binding fragments thereof are artificially generated.

In another related aspect of the present invention, there is provided antibody or antigen binding fragments thereof as herein above described with reference to all aspects for use in the manufacture of a medicament for a CD7+CD33+ hematological malignancy.

As used herein “a medicament” refers to a substance used for medical treatment (i.e. a medicine). The medicament may be, e.g. a T cell product that is for use in adoptive cell transfer.

As used herein CD7 is preferably human CD7 and CD33 is preferably human CD33. In certain embodiments, the bispecific antibodies or antigen fragments thereof specifically bind to CD7 and CD33 that are cell surface expressed. As used herein, the expression “cell surface-expressed” means one or more CD7 and/or CD33 protein(s) that is/are expressed on the surface of a cell in vitro or in vivo, such that at least a portion of a CD7 and/or a CD33 protein is exposed to the extracellular side of the cell membrane and is accessible to the bispecific antibody of antigen binding fragments thereof of the invention.

The term “CD7+CD33+ hematological malignancy” refers to a hematological malignancy characterized by the expression of both CD7 and CD33 on the surface of the malignant cells (e.g., a hematological malignancy that over expresses CD33 and/or CD7 on their cell surface and/or that express CD33 and/or CD7 at levels considered acceptable for therapy with the antibody or antigen binding fragments thereof that specifically binds to CD7 and CD33).

The antibody or antigen binding fragments thereof may be capable of inducing CD33 and/or CD7 receptor mediated internalization into a CD33+ and/or CD7+ cell.

CD7+CD33+ hematological malignancies include, but are not limited to, acute myeloid leukemia (AML), a myelodysplastic syndrome, a T-cell acute lymphoblastic leukemia, and a blastic plasmacytoid dendritic cell neoplasm (BPDCN).

The antibody or antigen fragments thereof may bispecifically binds CD33 and CD7 and wherein the CD33+ and CD7+ cell is an AML cell.

The antibody or antigen binding fragments thereof may be capable of mediating antibody dependent cellular cytotoxicity.

The antibody or antigen fragment thereof may be attached to, or formed with an immune effector cell. The immune effector cell may comprise a T cell and/or a NK cell. Preferably, immune effector cell is a T cell. The immune effector cell may be a bispecific anti-CD33 anti-CD7 CAR-T. The T cell may comprise a CD33+ T cell, a CD7+ T cell, or a combination thereof.

The antibody or antigen fragments thereof may comprise: i) a cell killing portion; ii) a CD7 binding portion; and iii) a CD33 binding portion.

In alternative embodiments, the CD33 and/or CD7 binding portion comprises an antigen binding fragments of an antibody.

The cell killing portion may be a cytotoxin and the skilled addressee will understand that a range of cytotoxins will be compatible with the composition. A cytotoxin may be selected from: i) a peptide toxin, ii) a chemical toxin, iii) an inhibitor of Bcl-2 or Bcl-axl, iv) an RNA Polymerase inhibitor such as a-amanitin, v) a spliceosome inhibitor, vi) a microtubule-targeting payload, or vii) a DNA-damaging payload. The antibody or antigen fragments thereof may further comprise a linking portion linking the cell kill portion with the CD7 binding portion and/or the CD33 binding portion. The antibody or antigen fragments thereof may be in the format of an antibody drug conjugate.

In the embodiment of a bispecific antibody, then such an antibody may be a full length antibody.

In a surprising finding the inventors have found that a bispecific antibody comprising a CD33 binding arm and a CD7 binding arm does not cause cytotoxicity in healthy human CD33 expressing myeloid cells. This therefore provides for the possibility of improved therapies for haematological malignancies, and in particular AML, without myelosuppression.

CD7 is a pan-leucocytic receptor expressed on progenitors of T and B lymphocytes, natural killer cells and dendritic cells (Hao, 2001; Sempowki, 1999) that plays an accessory role in T cell activation (Lazarovits, 1994; Stillwell, 2011) and persists on the surface of mature CD4<+>cells (Cotta, 2006; Lobac, 1985). CD7 has been widely studied as a target for delivery of cytotoxic molecules for leukaemia and lymphoma treatment (Peipp, 2002; Bremmer, 2006; Franker, 1997; Vallera, 1996; Waurzyniak, 1997).

CD33 is a 67 kDa plasma membrane protein that binds to sialic acid and is a member of the sialic acid-binding Ig-related lectin (SIGLEC) family of proteins. CD33 is known to be expressed on myeloid cells. CD33 expression has also been reported on a number of malignant cells.

Whilst CD33, a common myeloid antigen, is expressed on the majority of AML cells (De Propris, M. S., et al. (2011) High CD33 expression levels in acute myeloid leukemia cells carrying the nucleophosmin (NPM1) mutation, haematological, 96 1548-1551; Ehninger, A., et al. (2014) Distribution and levels of cell surface expression of CD33 and CD123 in acute myeloid leukemia, Blood Cancer Journal, 4 1-10) CD7, a common T and NK cell marker, is aberrantly expressed on a chemotherapy-resistant subpopulation (representing roughly 22%) of AML cells which confers a poor prognostic phenotype (Poeta, G. D., et al. (1995) CD7 Expression in Acute Myeloid Leukemia. Leuk. Lymphoma, 17, 111-119; Rohrs, S., et al. (2010) CD7 in acute myeloid leukemia: correlation with loss of wild-type CEBPA, consequence of epigenetic regulation, Journal of Hematology & Oncology, 3 1-7; Rausei-Mills, V., et al. (2008) Aberrant Expression of CD7 in Myeloblasts Is Highly Associated With De Novo Acute Myeloid Leukemias With FLT3/ITD Mutation, Am J Clin Pathol, 129 624-629; Shimamoto, T., et al. (1994) Clinical and Biological Characteristic of CD7+ Acute Myeloid Leukaemia, Cancer Genet Cytogenet 73 69-74; Reading, C. L., et al. (1993) Expression of unusual immunophenotype combinations in acute myelogenous leukemia, Blood 81 3083-3090; Ossenkoppele, G. J., et al (2011) Review of the relevance of aberrant antigen expression by flow cytometry in myeloid neoplasms British Journal of Haematology 153 421-436; Lo Coco, F., et al. (1989) CD7 positive acute myeloid leukaemia: a subtype associated with cell immaturity, British Journal of Haematology 73 480-485; Kita, K., et al. (1983) Clinical Importance of CD7 Expression in Acute Myelocytic Leukemia, Blood 81, 2399-2405; Eto, T., et al. (1992) Biological characteristics of CD7 positive acute myelogenous leukaemia, British lournal olHaernatology 82 508-511; Chang, H. (2004) Prognostic relevance of immunophenotyping in 379 patients with acute myeloid leukemia, Leukemia Research 28 43-48).

The CD7+ subtype of AML is associated with increased leucocytosis, poor response to chemotherapy and poor overall and disease-free survival (Kahl, C., et al. (2001) CD7+ and CD56+ Acute Myelogenous Leukemia is a Distinct Biologic and Clinical Disease Entity. Haematology and Blood Transfusion, 40 112-119). Clinically, CD7 AML patients are younger, more frequently males, have a higher incidence of central nervous system involvement and are often associated with less well differentiated subtypes of AML, further linked to poorer outcome (Tien, H. and Wang, C. (1998) CD7 Positive Hematopoietic Progenitors and Acute Myeloid Leukemia and other Minimally Differentiated Leukemia, Leukemia and Lymphoma, 3 93-98). The immaturity of the CD7+ AML cells has been further supported by the high expression of CD34 in this population. One study by Poeta et al, 1995, found that patients with CD7+ leukaemia had a significantly lower CR than those with the CD7- phenotype (32% versus 74%), indicating the degree of relapse and/or refractory nature of this subtype to standard of care (Poeta, G. D., et al. (1995) CD7 Expression in Acute Myeloid Leukemia. Leuk. Lymphoma, 17, 111-119).

CD7 is tightly correlated with and believed to be a hallmark of the FLT3-ITD+ AML subgroup. This subtype is associated with poorer clinical outcome due to deregulation of the FLT3 tyrosine kinase receptor, which signals to down regulate the translation of apoptotic proteins and as a result, induces resistance to chemotherapy-induced cell death in the AML cell population (Rausei-Mills, V., et al. (2008) Aberrant Expression of CD7 in Myeloblasts Is Highly Associated With De Novo Acute Myeloid Leukemias With FLT3/ITD Mutation, Am J Clin Pathol, 129 624-629). As a particularly poor prognostic subtype of AML, the FLT3 AML subgroup is a desirable disease class for novel drug developers to target, with many new therapeutics, including kinase inhibitors and monospecific ADCs, specifically targeting this population.

Several mechanisms have been described to explain the aberrant expression of CD7 in AML. These include disease-specific irregular gene expression in leukeamic cells (lineage infidelity), malignant transformation of pluripotent progenitor cells capable of lymphoid and myeloid differentiation or proliferation and maturation arrest of rare progenitor cells which may transiently express markers of different cell lineages during their normal cell differentiation (lineage promiscuity) (Tien, H. and Wang, C. (1998) CD7 Positive Hematopoietic Progenitors and Acute Myeloid Leukemia and other Minimally Differentiated Leukemia, Leukemia and Lymphoma, 3 93-98).

Transient CD7 expression has been reported in a subset of early progenitor cells capable of producing cells of both myeloid and lymphoid origin, but is lost during mature myeloid and lymphoid transformation (Tien, H. and Wang, C. (1998) CD7 Positive Hematopoietic Progenitors and Acute Myeloid Leukemia and other Minimally Differentiated Leukemia, Leukemia and Lymphoma, 3 93-98). In line with this, one study found that co-expression of these two antigens at low levels had been identified on certain subsets of healthy heamatopoietic cells, including pluripotent stem cells (CD33^{low}/CD7^{+/-}), some myeloid progenitor cells (CD33^{high}/CD7^{+/-}) and some T cell progenitors (CD33^{+/-}/CD7^{med}), but co-expression was lost during development (Barcena, A., et al. (1994) Tracing the Expression of CD7 and other Antigens during T- and Myeloid-cell Differentiation in the Human Fetal Liver and Thymus, Leukaemia and Lymphoma 17 1-11). It is therefore plausible that this co-expression pattern may be a result of the clonal expansion of a certain subset of progenitor cells, captured at a particular stage in development whereby these two antigens are transiently seen together and this expression is amplified during the malignant transformation.

As used herein, the terms “treat”, “treating” and “treatment” are taken to include an intervention performed with the intention of preventing the development or altering the pathology of a disorder or symptom. Accordingly, “treatment” refers to both therapeutic treatment and prophylactic or preventative measures, wherein the object is to prevent or slow down (lessen) the targeted disorder or symptom. Accordingly, the term “treating” encompasses treating and/or preventing the development of a disorder or symptom. As used herein, “therapy” refers to the prevention or treatment of a disease or disorder. Therapy may be prophylactic or therapeutic.

In such aspects, the antibody or antigen binding fragments thereof of the invention are administered to a patient in remission from the hematological malignancy, resulting in preventing or delaying recurrence of the underlying hematological malignancy.

As used herein, a “patient” is typically a human who is undergoing treatment for, or has been diagnosed as having, hematological malignancy, preferably a CD7+CD33+ hematological malignancy. In some embodiments, the antibody or antigen binding fragments thereof are administered to a patient in remission from CD7+CD33+ hematological malignancy, whereby the recurrence of the hematological malignancy is prevented or delayed. In some embodiments, the patient lacks detectable cells of the hematological malignancy. As used herein, a “lack of detectable cells” is determined by standard diagnostic or prognostic methods. A patient in remission from AML typically exhibits resolution of abnormal clinical features, return to normal blood counts and normal hematopoiesis in the bone marrow with <5% blast cells, a neutrophil count of >1.000-1,500, a platelet count of >100,000, and disappearance of the leukemic clone. See, e.g., The Merck Manual, Sec. 11, Ch. 138 (17th ed. 1997): Estey, 2001, Cancer 92(5): 1059-1073.

In some embodiments, the patient in remission from the CD7+CD33+ hematological malignancy has not undergone a bone marrow transplant. In other embodiments, the patient in remission from the CD7+CD33+ hematological malignancy has undergone a bone marrow transplant. The bone marrow transplant can be either an autologous or an allogeneic bone marrow transplant.

In embodiments treating a CD7+CD33+ hematological malignancy (for example AML) and delaying preventing or delaying recurrence of CD7+CD33+ hematological malignancy (for example AML) involves the inducing AML cancer cell death and/or inhibiting AML cancer cell growth.

The antibody or antigen binding fragments thereof may be part of a composition (e.g. a therapeutic composition) that comprises the compound (i.e. the antibody or antigen binding fragments thereof) and one or more other components. A composition may be a therapeutic composition that comprises the the antibody or antigen binding fragments thereof and a pharmaceutically acceptable excipient, adjuvant, diluent and/or carrier. Therapeutic compositions may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected compound without causing any undesirable biological effects or interacting in a deleterious manner with any of the other components of the pharmaceutical composition in which it is contained.

Excipients are natural or synthetic substances formulated alongside an active ingredient (e.g. the vaccine, cell cycle inhibitor, modulator of an immune suppression mechanism, or immune check point inhibitor (as appropriate)), included for the purpose of bulking-up the formulation or to confer a therapeutic enhancement on the active ingredient in the final dosage form, such as facilitating drug absorption or solubility. Excipients can also be useful in the manufacturing process, to aid in the handling of the active substance concerned such as by facilitating powder flowability or non-stick properties, in addition to aiding in vitro stability such as prevention of denaturation over the expected shelf life. Pharmaceutically acceptable excipients are well known in the art. A suitable excipient is therefore easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well known in the art. A suitable adjuvant is therefore easily identifiable by one of ordinary skill in the art.

Diluents are diluting agents. Pharmaceutically acceptable diluents are well known in the art. A suitable diluent is therefore easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is therefore easily identifiable by one of ordinary skill in the art

As used herein, the terms “effective amount” and “therapeutically effective amount” refer to the quantity of the active therapeutic agent sufficient to yield a desired therapeutic response without undue adverse side effects such as toxicity, irritation, or allergic response. The specific “effective amount” will, obviously, vary with such factors as the particular condition being treated, the physical condition of the patient, the type of animal being treated, the duration of the treatment, the nature of concurrent therapy (if any), and the specific formulations employed and the structure of the compounds or its derivatives. In this case, an amount would be deemed therapeutically effective if it resulted in one or more of, but not limited to, the following: (a) the inhibition of cancer cell growth (e.g. AML cells); and (b) the killing of cancer cells (e.g. AML cells).

The dose of the antibody or antigen binding fragments thereof and therapeutic compositions thereof administered to a patient may vary depending upon the age and the size of the patient, target disease, conditions, route of administration, and the like. The preferred dose is typically calculated according to body weight or body surface area.

Methods of administration of the antibody or antigen binding fragments thereof and therapeutic compositions thereof include, but are not limited to, intradermal, intramuscular, intraperitoneal, intravenous, subcutaneous, intranasal, epidural, and oral routes. The antibody or antigen binding fragments thereof and therapeutic compositions thereof may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Preferably, the dual targeting therapy described herein will provide a benefit to the treatment of a CD7+CD33+ hematological malignancy in a subject in need thereof. For example, the dual targeting therapy may have an additive or synergistic effect on the treatment of AML in a subject in need thereof. A dual targeting therapy is defined as affording an “additive effect”, “synergistic effect” or a “synergistic treatment” if the effect is therapeutically superior, as measured by, for example, the extent of the response (e.g. apoptosis or cell viability), the response rate, the time to disease progression or the survival period, to that achievable on dosing one or other of the components of the dual targeting therapy at its conventional dose. For example, the effect of the dual targeting therapy is additive if the effect is therapeutically superior to the effect achievable with an antibody or antigen binding fragments thereof that specifically binds to CD33 or CD7 alone. For example, the effect of the combination treatment may be synergistic if the effect of the combination treatment supersedes the effect of the individual treatments added together. Further, the effect of the combination is beneficial (e.g. additive or synergistic) if a beneficial effect is obtained in a group of subjects that does not respond (or responds poorly) to a cell-inhibiting agent that specifically binds to CD33 alone or a cell-inhibiting agent that specifically binds to CD7 alone. In addition, the effect of the combination treatment is defined as affording a benefit (e.g. additive or synergistic effect) if one of the components is dosed at its conventional dose and the other component is dosed at a reduced dose and the therapeutic effect, as measured by, for example, the extent of the response, the response rate, the time to disease progression or the survival period, is equivalent to or better than that achievable on dosing conventional amounts of either one of the components of the combination treatment.

As used herein, “killing of a target cell” relates to an inhibition of protein synthesis, for example such that cell viability is reduced, or an induction of apoptosis resulting in elimination or death of target cells. Assays to determine cell killing and apoptosis are well known in the art. Cytotoxicity assays assess the number of live and dead cells in a population after treatment with a pharmacological substance (e.g. an LDH cytotoxicity assay, or a live-dead cell assay). Apoptosis assays assess how cells are dying by measuring markers that are activated upon cell death (e.g. a PS exposure assay, a caspase activation assay, a DNA fragmentation assay, a GSH/GSSG determination, a LDH cytotoxicity assay, a live-dead cell assay, or a non-caspase protease activation assay).

As used herein “inhibit the cell growth” (e.g., referring to target cells) refers to any measurable decrease in the growth or proliferation of a target cell when contacted with the antibody or antigen binding fragments thereof according to the present invention as compared to the growth of the same cell not in contact with the antibody or antigen binding fragments thereof according to the present disclosure, e.g., the inhibition of growth of a cell by at least about 10%, 20%, 30%, 40%, 50%, 60%, 70%, 80%, 90%, 99%, or 100%. Assays to determine cell viability or proliferation are well known in the art. Cell viability assays assess how healthy the cells are by measuring markers of cellular activity (e.g. an ATP and ADP determination assay, a cell cycle assay, a cell proliferation assay, a cell viability assay, an LHD cytotoxicity assay, or a live-dead cell assay). Cell proliferation assays assess the growth rate of a cell population or to detect daughter cells in a growing population (e.g. a cell cycle assay, a cell proliferation assay, a cell viability assay, or a senescence assay).

As used herein, “CD33 expressing cell” and “CD33+ cell” refers to a cell with CD33 as surface antigen. As used herein, “CD7 expressing cell” and “CD7+ cell” refers to a cell with CD7 as surface antigen. As used herein, “CD33 and CD7 expressing cell” and “CD33+CD7+ cell” refers to a cell with both CD33 and CD7 as surface antigens.

As used herein “target cell” refers to a cell or cell-type characterized by the expression or overexpression of the target molecule CD7 and CD33. Any type of cell expressing CD7 and CD33 may be envisaged as a target cell for treatment with the antibody or antigen binding fragments thereof of the invention. In certain embodiments, the cell is a tumour cell, for example a tumour cell from a hematological malignancy, such as an AML cell.

In certain embodiments, the antibody or antigen binding fragments thereof described herein are capable of inducing CD33 receptor mediated internalization of said antibody or antigen binding fragments thereof into a CD33+ cell, and/or CD7 receptor mediated internalization of said the antibody or antigen binding fragments thereof into a CD7+ cell. In certain embodiments, the antibody or antigen binding fragments thereof is an antibody or antigen binding fragments thereof that specifically binds to both CD33 and CD7 and is capable of inducing internalization of the agent into a CD7+CD33+ cell upon binding of both CD7 and CD33 on a cell surface.

As used herein, “CD33 receptor mediated internalization” refers to taken up by (i.e., entry of) a CD33+ cell upon binding to CD33 on the cell surface. For therapeutic applications, internalization in vivo is contemplated. As used herein, “CD7 receptor mediated internalization” refers to taken up by (i.e., entry of) a CD7+ cell upon binding to CD7 on the cell surface. For therapeutic applications, internalization in vivo is contemplated.

For therapeutic applications, the number of the antibody or antigen binding fragments thereof internalized will be sufficient or adequate to kill an CD33+CD7+ cell, especially an CD7+CD33+ hematological cancer cell, such as an AML cell. Depending on the potency of the antibody or antigen binding fragments thereof, in some instances, the uptake of a single molecule into the cell is sufficient to kill the target cell to which the agent binds.

In certain embodiments, the antibody or antigen binding fragments thereof of the invention may be ADC's, small-molecule drug conjugates (SMDCs), immunotoxins, peptide and non-peptide conjugates, imaging agents, therapeutic vaccines, nanoparticles.

The terms “antibody” or “antibodies” as used herein refer to molecules or active fragments of molecules that bind to known antigens, particularly to immunoglobulin molecules and to immunologically active portions of immunoglobulin molecules, i.e. molecules that contain a binding site that immunospecifically binds an antigen (i.e. CD7 or CD33). The immunoglobulin according to the invention can be of any class (IgG, IgM, IgD, IgE, IgA and IgY) or subclass (e.g. IgG1, IgG2, IgG3, IgG4, IgA1 and IgA2) or subclasses (isotypes) of immunoglobulin molecule (e.g. IgG in IgG1, IgG2, IgG3, and IgG4, or IgA in IgA1 and IgA2).

Within the scope of the present invention the terms “antibody” or “antibodies” include human and humanized antibodies as well as active fragments thereof. Examples of active fragments of molecules that bind to known antigens include Fab, F(ab′), F(ab′)², scFv and Fv fragments, including the products of a Fab immunoglobulin expression library and epitope-binding fragments of any of the antibodies and fragments mentioned above.

As used herein the term “humanized antibody” or “humanized version of an antibody” refers to antibodies in which the framework or “complementarity determining regions” (CDR) have been modified to comprise the CDR of an immunoglobulin of different specificity as compared to that of the parent immunoglobulin. In some exemplary embodiments, the CDRs of the VH and VL are grafted into the framework region of human antibody to prepare the “humanized antibody.” See e.g. Riechmann, L., et al., Nature 332 (1988) 323-327; and Neuberger, M. S., et al., Nature 314 (1985) 268-270. The heavy and light chain variable framework regions can be derived from the same or different human antibody sequences. The human antibody sequences can be the sequences of naturally occurring human antibodies. Human heavy and light chain variable framework regions are listed e.g. in Lefranc, M.-P., Current Protocols in Immunology (2000)—Appendix 1P A.1P.1-A.1P.37 and are accessible via IMGT, the international ImMunoGeneTics information System® (http://imgt.cines.fr) or via http://vbase.mrc-cpe.cam.ac.uk, for example. Optionally the framework region can be modified by further mutations. Exemplary CDRs correspond to those representing sequences recognizing the antigens noted above for chimeric antibodies. In some embodiments, such humanized version is chimerized with a human constant region. The term “humanized antibody” as used herein also comprises such antibodies which are modified in the constant region to generate the properties according to the disclosure, especially in regard to C1q binding and/or FcR binding, e.g. by “class switching” i.e. change or mutation of Fc parts (e.g. from IgG1 to IgG4 and/or IgG1/IgG4 mutation).

As used herein the term “human antibody” is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice results in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Brueggemann, M. D., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole, A., et al. and Boerner, P., et al. are also available for the preparation of human monoclonal antibodies (Cole, A., et al., Monoclonal Antibodies and Cancer Therapy, Liss, A. R. (1985) p. 77; and Boerner, P., et al., J. Immunol. 147 (1991) 86-95). As already mentioned, according to the instant disclosure the term “human antibody” as used herein also comprises such antibodies which are modified in the constant region to generate the properties according to the disclosure, for example in regard to C1q binding and/or FcR binding, e.g. by “class switching” i.e. change or mutation of Fc parts (e.g. from IgG1 to IgG4 and/or IgG1/IgG4 mutation).

As used herein the term “antibody fragment” refers to a portion of a full length antibody, the term “antigen binding fragments” refers to a variable domain thereof, or at least an antigen binding site thereof, for example the CDRs. Examples of antibody fragments include diabodies, single-chain antibody molecules, and multispecific antibodies formed from antibody fragments. scFv antibodies are, e.g., described in Huston, J. S., Methods in Enzymol. 203 (1991) 46-88. Antibody fragments can be derived from an antibody of the present invention by a number of art-known techniques. For example, purified monoclonal antibodies can be cleaved with an enzyme, such as pepsin, and subjected to HPLC gel filtration. The appropriate fraction containing Fab fragments can then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

As used herein the term “bispecific antibodies” refers to antibodies that bind to two (or more) different antigens. A bispecific antibody typically comprises at least two different variable domains, wherein each variable domain is capable of specifically binding to a separate antigen. In certain aspects, the bispecific antibodies of the invention are human antibodies. As used herein, the expression “bispecific antigen-binding molecule” means a protein, polypeptide or molecular complex comprising at least a first antigen-binding domain and a second antigen-binding domain. Each antigen-binding domain within the bispecific antigen-binding molecule comprises at least one CDR that alone, or in combination with one or more additional CDRs, specifically binds to a particular antigen. In the context of the present invention, the first antigen-binding domain specifically binds a first antigen (e.g., CD7), and the second antigen-binding domain specifically binds a second, distinct antigen (e.g., CD33). In certain aspects, the bispecific molecules are capable of simultaneously binding to human CD7 and human CD33.

In certain embodiments the bispecific antibodies may be referred to as “anti-CD7xCD33” or “anti-CD7/anti-CD33” and so forth.

Any bispecific antibody format or technology may be used to make the bispecific antigen-binding molecules of the present invention. Specific exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1 /IgG2, dual acting Fab (DAF)-IgG, Mab2 bispecific formats (see, e.g., Klein et al. 2012, imAbs 4:6, 1-1 1 , and references cited therein, for a review of the foregoing formats) and Fab-based bispecific formats. In certain embodiments, the bispecific antibody is a Fab-based anti-CD7xCD33 bispecific.

As used herein the term “specific” and “specifically” are used interchangeably to indicate that biomolecules other than CD7 or CD33 (or where the biomolecule is a bispecific molecule both CD7 and CD33) do not significantly bind to the antibody. In some embodiments, the level of binding to a biomolecule other than CD7 or CD33 is negligible (e.g., not determinable) by means of ELISA or an affinity determination.

By “negligible binding” a binding is meant, which is at least about 85%, particularly at least about 90%, more particularly at least about 95%, even more particularly at least about 98%, but especially at least about 99% and up to 100% less than the binding to CD7 or CD33.

The binding affinity of an antibody to a peptide or epitope may be determined with a standard binding assay, such as surface plasmon resonance technique (BIAcore®, GE-Healthcare Uppsala, Sweden). The term “surface plasmon resonance,” as used herein, refers to an optical phenomenon that allows for the analysis of real-time biospecific interactions by detection of alterations in protein concentrations within a biosensor matrix, for example using the BlAcore system (Pharmacia Biosensor AB, Uppsala, Sweden and Piscataway, N. J.). For further descriptions, see Jonsson, U., et al. (1993) Ann. Biol. Clin. 51 : 19-26; Jonsson, U., et al. (1991) Biotechniques 11 :620-627; Johnsson, B., et al. (1995) J. Mol. Recognit. 8: 125-131; and Johnnson, B., et al. (1991) Anal. Biochem. 198:268-277.

In one embodiment, the antibody or antigen binding fragments thereof of the invention are capable of mediating antibody dependent cell cytotoxicity. Antibody dependent cellular cytotoxicity (ADCC) is an immune effector cell mediated mechanism which may contribute to anti-tumor activity of monoclonal antibodies (Weiner GJ. Monoclonal antibody mechanisms of action in cancer. Immunol Res. 2007,39(I-3):271-8). The relevance of ADCC for anti-tumor efficacy has been demonstrated in preclinical models, e.g. in mouse tumor models (e.g. Clynes RA, Towers TL, Presta LG, Ravetch JV. Inhibitory Fc receptors modulate in vivo cytoxicity against tumor targets. Nat Med. 2000 Apr;6(4):443-6). Data from clinical trials support the relevance of ADCC for clinical efficacy of therapeutic antibodies (e.g. Weng WK, Levy R Two immunoglobulin G fragment C receptor polymorphisms independently predict response to rituximab in patients with follicular lymphoma. J Clin Oncol. 2003 November I;21(21):3940-7. Epub 2003 Sep. 15). Interactions of monoclonal antibodies with Fc receptors on immune cells contribute to ADCC. The Fc of antibodies can be modified in order to display enhanced affinity to Fc receptors (e.g. Presta LG Engineering of therapeutic antibodies to minimize immunogenicity and optimize function. Adv Drug Deliv Rev. 2006 Aug. 7;58(5-6):640-56. Epub 2006 May 23). Such enhanced affinity to Fc receptors results in increased ADCC activity which may lead to increased anti-tumor efficacy in patients.

In an alternative embodiment, the antigen binding fragments thereof of the invention are immunoresponsive cells which expresses a chimeric antigen T cell receptor protein (CAR), wherein the chimeric T cell receptor protein specifically binds to CD7 and CD33. In one embodiment immunoresponsive cell is bispecific and which a chimeric antigen T cell receptor protein (CAR), wherein the chimeric T cell receptor protein specifically binds to CD7 and a chimeric antigen T cell receptor protein (CAR), wherein the chimeric T cell receptor protein specifically binds to CD33. The immunoresponsive cell expressing the CAR may be selected from the group consisting of a T cell, a hematopoietic stem cell, a natural killer cell, a natural killer T cell, a B cell and a cell of monocytic lineage. In a particular embodiment, the immunoresponsive cell is a T cell.

In some embodiments, the immunoresponsive cell is autologous to the subject. In another embodiment, the immunoresponsive cell is not autologous to the subject. In a particular embodiment, the immunoresponsive cell is a T cell and is autologous to the subject to be treated.

In some embodiments, the antibody or antigen binding fragments thereof comprises a binding portion (i.e. a CD33 binding portion, a CD7 binding portion, or a CD7 and a CD33 binding portion) and a cell killing portion. In certain embodiments, the cell binding portion is an antibody or antigen binding fragments thereof. In particular embodiments the cell binding portion is an antibody or antigen binding fragments thereof.

In some embodiments, the antibody or antigen binding fragments thereof further comprises (or is incorporated or associated with) a cytotoxic or cytostatic agent, i.e. a compound that kills or inhibits tumour cells. Such agents may impart their cytotoxic and cytostatic effects by mechanisms including tubulin binding, DNA binding, proteasome and/or topoisomerase inhibition.

The cytotoxic or cytostatic agent may be, for example, a peptide toxin, a small molecule toxin or a radioisotope.

In one embodiment the cytotoxic or cytostatic agent may be a tubulin inhibitor; or a DNA interacting agent. Tubulin inhibitors modulate tubulin polymerization. DNA interacting agents target cellular DNA.

In an embodiment the cytotoxic or cytostatic agent is a tubulin inhibitor. In an embodiment, the tubulin inhibitor is selected from the group consisting of: (a) an auristatin; and (b) a maytansine derivative. In an embodiment, the cytotoxic or cytostatic agent is an auristatin. Auristatins include synthetic derivatives of the naturally occurring compound Dolastatin-10. Auristatins are a family of antineoplastic/cytostatic pseudopeptides. Dolastatins are structurally unique due to the incorporation of 4 unusual amino acids (Dolavaine, Dolaisoleuine, Dolaproine and Dolaphenine) identified in the natural biosynthetic product. In addition, this class of natural product has numerous asymmetric centres defined by total synthesis studies by Pettit et al (U.S. Pat. No. 4,978,744). It would appear from structure activity relationships that the Dolaisoleuine and Dolaproine residues appear necessary for antineoplastic activity (U.S. Pat. Nos. 5,635,483 and 5,780,588). In an embodiment, the auristatin is selected from the group consisting of: Auristatin E (AE); Monomethylauristatin E (MMAE); Auristatin F (MMAF); vcMMAE; vcMMAF; mcMMAE and mcMMAF. In an embodiment, the cytotoxic or cytostatic agent is a maytansine or a structural analogue of maytansine. In an embodiment, the cytotoxic or cytostatic agent is a maytansine. Maytansines include structurally complex antimitotic polypetides. Maytansines are potent inhibitors of microtubulin assembly which leads towards apoptosis of tumour cells. In an embodiment the maytansine is selected from the group consisting of: Mertansine (DM1); and a structural analogue of maytansine such as DM3 or DM4. Preferably, the drug is mertansine (DM1).

In an embodiment, the cytotoxic or cytostatic agent is DNA interacting agent. In an embodiment, the DNA interacting agent is selected from the group consisting of: (a) calicheamicins, (b) duocarmycins and (c) pyrrolobenzodiazepines (PBDs). In an embodiment, the cytotoxic or cytostatic agent is a calicheamicin. Calicheamicin is a potent cytotoxic agent that causes double-strand DNA breaks, resulting in cell death. Calicheamicin is a naturally occurring enediyne antibiotic (A. L. Smith et al, J. Med. Chem., 1996, 39,11, 2103-2117). Calicheamicin was found in the soil microorganism Micromonosporaechinospora. In an embodiment, the calicheamicin is calicheamicin gamma 1. In an embodiment, the drug is a duocarmycin. Duocarmycins are potent anti-tumour antibiotics that exert their biological effects through binding sequence-selectively in the minor groove of DNA duplex and alkylating the N3 of adenine (D. Boger, Pure & Appl. Chem., 1994, 66, 4, 837-844). In an embodiment, the duocarmycin is selected from the group consisting of: Duocarmycin A; Duocarmycin B1; Duocarmycin B2; Duocarmycin C1; Duocarmycin C2; Duocarmycin D; Duocarmycin SA; Cyclopropylbenzoindole (CBI) duocarmycin; Centanamycin; Rachelmycin (CC-1065); Adozelesin; Bizelesin; and Carzelesin. In an embodiment, the cytotoxic or cytostatic agent is a pyrrolobenzodiazepine. Pyrrolobenzodiazepines (PBDs) are a class of naturally occurring anti-tumour antibiotics. Pyrrolobenzodiazepines are found in Streptomyces. PBDs exert their anti-tumour activity by covalently binding to the DNA in the minor groove specifically at purine-guanine-purine units. They insert on to the N2 of guamine via an aminal linkage and, due to their shape, they cause minimal disruption to the DNA helix. It is believed that the formation of the DNA-PBD adduct inhibits nucleic acid synthesis and causes excision-dependent single and double stranded breaks in the DNA helix. As synthetic derivatives the joining of two PBD units together via a flexible polymethylene tether allows the PBD dimers to cross-link opposing DNA strands producing highly lethal lesions. In an embodiment, the cytotoxic or cytostatic agent is a synthetic derivative of two pyrrolobenzodiazepines units joined together via a flexible polymethylene tether. In an embodiment, the pyrrolobenzodiazepine is selected from the group consisting of: Anthramycin (and dimers thereof); Mazethramycin (and dimers thereof); Tomaymycin (and dimers thereof); Prothracarcin (and dimers thereof); Chicamycin (and dimers thereof); Neothramycin A (and dimers thereof); Neothramycin B (and dimers thereof); DC-81 (and dimers thereof); Sibiromycin (and dimers thereof); Porothramycin A (and dimers thereof); Porothramycin B (and dimers thereof); Sibanomycin (and dimers thereof); Abbeymycin (and dimers thereof); SG2000; and SG2285.

In an embodiment, the cytotoxic or cytostatic agent is a drug that targets DNA interstrand crosslinks through alkylation. A drug that targets DNA interstrand crosslinks through alkylation is selected from: a DNA targeted mustard; a guanine-specific alkylating agent; and a adenine-specific alkylating agent. In an embodiment, the cytotoxic or cytostatic agent is a DNA targeted mustard. For example, the DNA targeted mustard may be selected from the group consisting of: an oligopyrrole; an oligoimidazole; a Bis-(benzimidazole) carrier; a Polybenzamide Carrier; and a 9-Anilinoacridine-4-carboxamide carrier.

In an embodiment, the cytotoxic or cytostatic agent is selected from the group consisting of: Netropsin; Distamycin; Lexitropsin; Tallimustine; Dibromotallimustine; PNU 157977; and MEN 10710.

In an embodiment, the cytotoxic or cytostatic agent is a Bis-(benzimidazole) carrier. Preferably, the drug is Hoechst 33258.

A guanine-specific alkylating agent is a highly regiospecific alkylating agents that reacts at specific nucleoside positions. In an embodiment, the cytotoxic or cytostatic agent is a guanine-specific alkylating agent selected from the group consisting of: a G-N2 alkylators; a A-N3 alkylator; a mitomycin; a carmethizole analogue; a ecteinascidin analogue. In an embodiment, the mitomycin is selected from: Mitomycin A; Mitomycin C; Porfiromycin; and KW-2149. In an embodiment, the a carmethizole analogue is selected from: Bis-(Hydroxymethyl)pyrrolizidine; and NSC 602668. In an embodiment, the ecteinascidin analogue is Ecteinascidin 743.

Adenine-specific alkylating agents are regiospecific and sequence-specific minor groove alkylators reacting at the N3 of adenines in polypyrimidines sequences.

Cyclopropaindolones and duocamycins may be defined as adenine-specific alkylators. In an embodiment, the cytotoxic or cytostatic agent is a cyclopropaindolone analogue. Preferably, the drug is selected from: adozelesin; and carzelesin.

In an embodiment, the cytotoxic or cytostatic agent is a benz[e]indolone. Preferably, the cytotoxic or cytostatic agent is selected from: CBI-TMI; and iso-CBI.

In an embodiment, the cytotoxic or cytostatic agent is bizelesin. In an embodiment, the cytotoxic or cytostatic agent is a Marine Antitumor Drug. Marine Antitumor Drugs has been a developing field in the antitumor drug development arena (I. Bhatnagaret a/,Mar. Drugs 2010, 8, P2702-2720 and T. L. Simmons et al, Mol. Cancer Ther. 2005, 4(2), P333-342). Marine organisms including sponges, sponge-microbe symbiotic association, gorgonian, actinomycetes, and soft coral have been widely explored for potential anticancer agents.

In an embodiment, the cytotoxic or cytostatic agent is selected from: Cytarabine, Ara-C; Trabectedin (ET-743); and EribulinMesylate. In an embodiment, the EribulinMesylate is selected from: (E7389); Soblidotin (TZT 1027); Squalamine lactate; CemadotinPlinabulin (NPI-2358); Plitidepsin; Elisidepsin; Zalypsis; Tasidotin, Synthadotin; (ILX-651); Discodermolide; HT1286; LAF389; Kahalalide F; KRN7000; Bryostatin 1; Hemiasterlin (E7974); Marizomib; Salinosporamide A; NPI-0052); LY355703; CRYPTO 52; Depsipeptide (NSC630176); Ecteinascidin 743; Synthadotin; Kahalalide F; Squalamine; Dehydrodidemnin B; Didemnin B; Cemadotin; Soblidotin; E7389; NVP-LAQ824; Discodermolide; HTI-286; LAF-389; KRN-7000 (Agelasphin derivative); Curacin A; DMMC; Salinosporamide A; Laulimalide; Vitilevuamide; Diazonamide; Eleutherobin; Sarcodictyin; Peloruside A; Salicylihalimides A and B; Thiocoraline; Ascididemin; Variolins; Lamellarin D; Dictyodendrins; ES-285 (Spisulosine); and Halichondrin B.

The following cytotoxic or cytostatic agent are also encompassed by the present invention: Amatoxins (a-amanitin)- bicyclic octapeptides produced by basidiomycetes of the genus Amanita,e.g. the Green Deathcap mushroom; Tubulysins; Cytolysins; dolabellanins; Epothilone A, B, C, D, E, F. Epothilones—constitute a class of non-taxane tubulin polymerisation agents and are obtained by natural fermentation of the myxobacteriumSorangiumcellulosum. These moieties possess potent cytotoxic activity which is linked to the stabilisation of microtubules and results in mitotic arrest at the G2/M transition. Epothilones have demonstrated potent cytotoxicity across a panel of cancer cell lines and has often exhibited greater potency than paclitaxel (X. :Pivot et al, European Oncology,2008;4(2), P42-45). In an embodiment, the drug is amatoxin. In an embodiment, the drug is tubulysin. In an embodiment, the drug is cytolysin. In an embodiment, the drug is dolabellanin. In an embodiment, the drug is epothilone.

The following cytotoxic or cytostatic agent are also encompassed by the present invention. In an embodiment, the drug is selected from: Doxorubicin; Epirubicin; Esorubicin; Detorubicin; Morpholino-doxorubicin; Methotrexate; Methopterin; Bleomycin; Dichloromethotrexate; 5-Fluorouracil; Cytosine-β-D-arabinofuranoside; Taxol; Anguidine; Melphalan; Vinblastine; Phomopsin A; Ribosome-inactivating proteins (RIPs); Daunorubicin; Vinca alkaloids; Idarubicin; Melphalan; Cis-platin; Ricin; Saporin; Anthracyclines; Indolino-benzodiazepines; 6-Mercaptopurine; Actinomycin; Leurosine; Leurosideine; Carminomycin; Aminopterin; Tallysomycin; Podophyllotoxin; Etoposide; Hairpin polyamides; Etoposide phosphate; Vinblastine; Vincristine; Vindesine; Taxotere retinoic acid; N8-acetyl spermidine; Camptothecin; Esperamicin; and Ene-diynes.

In one embodiment, the cell killing portion is a peptide toxin, for example an auristatin such as MMAE. In one embodiment, the antibody or antigen binding fragments thereof comprises a binding portion and a cell killing portion, wherein the binding portion is an anti-CD7 anti-CD33 bispecific antibody or binding portion thereof and wherein the cell killing portion is a peptide toxin, for example an auristatin such as MMAE.

In certain embodiments, the antibody or antigen binding fragments thereof comprises a binding portion that is conjugated to a cell killing portion. Such conjugates may be prepared by in vitro methods known to one of ordinary skill in the art. Techniques for conjugating cytotoxic or cytostatic agent to proteins, and in particular to antibodies, are well-known. (See, e.g., Alley et ah, Current Opinion in Chemical Biology 2010 14: 1-9; Senter, Cancer J., 2008, 14(3): 154-169.)

In certain embodiments, a linking group is used to conjugate the binding portion and the cell killing portion.

The linker can be cleavable under intracellular conditions, such that cleavage of the linker releases the cell killing portion from the binding portion in the intracellular environment. The cleavable linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in NTB-A-expressing cells. For example, a peptidyl linker that is cleavable by the thiol-dependent protease cathepsin-B, which is highly expressed in cancerous tissue, can be used {e.g., a linker comprising a Phe-Leu or a Val-Cit peptide).

The cleavable linker can be pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used.

Other linkers are cleavable under reducing conditions (e.g., a disulfide linker). The cleavable linker can also be a malonate linker (Johnson et al, Anticancer Res. 15 :1387-93, 1995), a maleimidobenzoyl linker (Lau et al, Bioorg-Med-Chem. 3: 1299-1304, 1995), or a 3′-N-amide analog (Lau et al, Bioorg-Med-Chem. 3: 1305-12, 1995).

In some embodiments the linker can be a protease cleavable linker, for example a valine-citrulline, which may be cleaved by cathepsin B in the lysosome.

The linker also can be a non-cleavable linker, such as an maleimido-alkylene- or maleimide-aryl linker that is directly attached to the therapeutic agent and released by proteolytic degradation of the binding portion.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### DETAILED DESCRIPTION OF THE INVENTION

Embodiments of the invention are described below, by way of example only with reference to and as illustrated in the following figures:

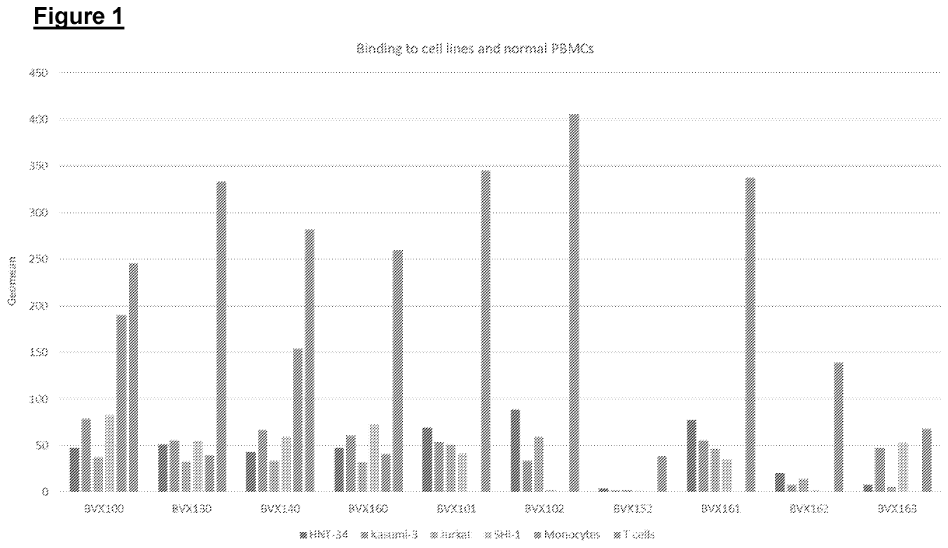

FIG. 1 is a bar graph that shows the binding of affinity reduced bi-Fabs to CD33+/CD7+ cell lines, normal PBMC monocytes and T cells isolated from healthy human donors at 10 nM. 50000 cells of HNT-34 (CD33+/CD7+), Kasumi-3 (CD33+/CD7+), Jurkat (CD33-/CD7+), SHI-1 (CD33+/CD7−), healthy PBMC monocytes (CD33+/CD7−) and healthy T cells (CD33−/CD7+) were tested per CD33+/CD7+ bi-Fab antibody. The cells were resuspended in 100 μl of 10 nM bi-Fab on ice for 1 hour. Following incubation, the cells were pelleted and incubated on ice with 50 μl of Mouse anti-Human IgG Fab Secondary Antibody, PE diluted 1 in 17 in ice cold PBS/0.1% BSA for a further hour. Cells were washed and resuspended in PBS for analysis on the FACS Calibur. Data was plotted in excel;

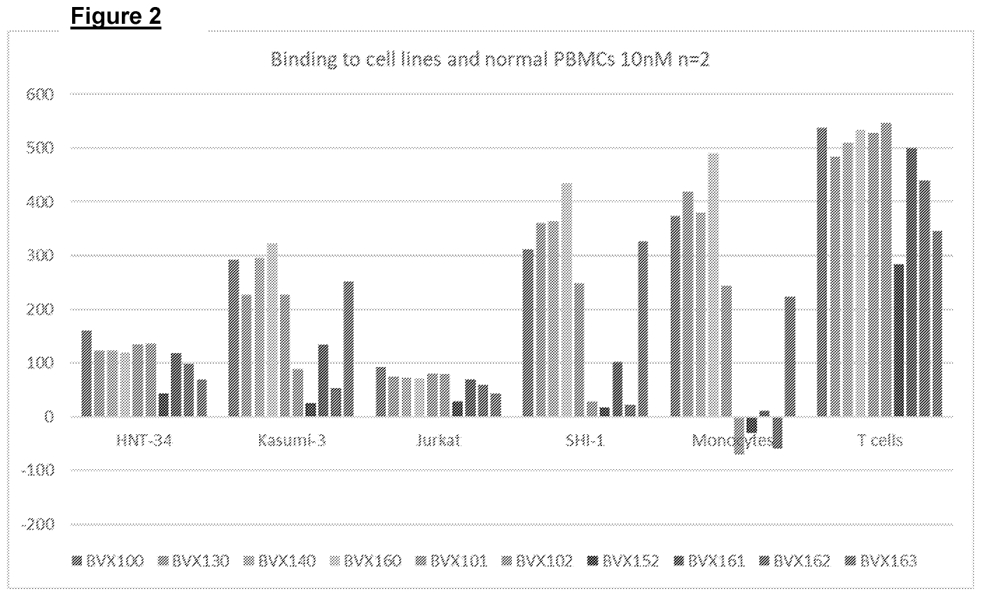

FIG. 2 is a bar graph that shows the binding of each affinity reduced bi-Fab to CD33+/CD7+ cell lines, normal PBMC monocytes and T cells isolated from healthy human donors at 10 nM. 50000 cells of HNT-34 (CD33+/CD7+), Kasumi-3 (CD33+/CD7+), Jurkat (CD33−/CD7+), SHI-1 (CD33+/CD7−), healthy PBMC monocytes (CD33+/CD7−) and healthy T cells (CD33−/CD7+) were tested per CD33+/CD7+ bi-Fab antibody. The cells were resuspended in 100 μl of 10 nM bi-Fab on ice for 1 hour. Following incubation, the cells were pelleted and incubated on ice with 50 μl of Mouse anti-Human IgG Fab Secondary Antibody, PE diluted 1 in 17 in ice cold PBS/0.1% BSA for a further hour. Cells were washed and resuspended in PBS for analysis on the FACS Calibur. Data was plotted in excel;

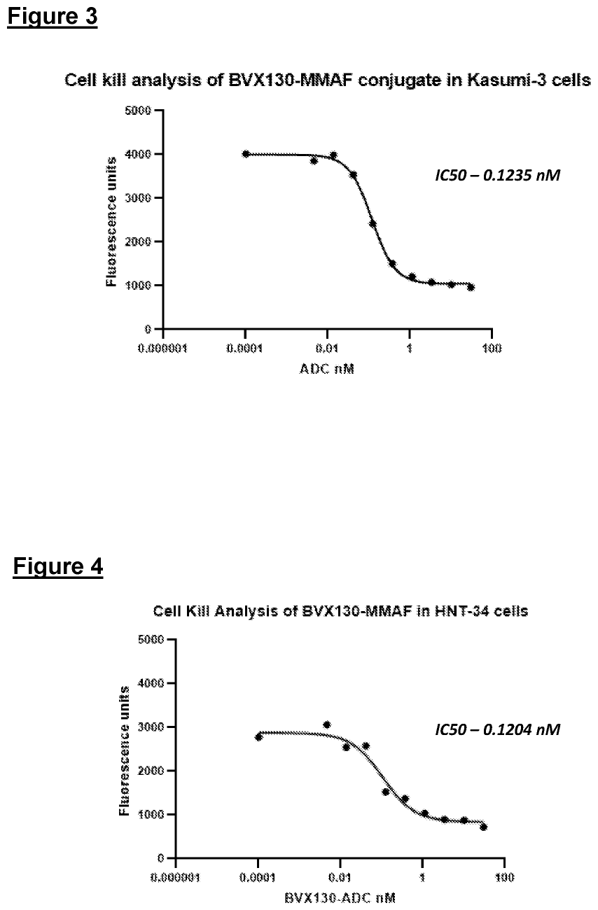

FIG. 3 is a graph describing a cell kill assay conducted using a 10-point dose response of directly conjugated BVX130-MMAF on 2×10⁴ Kasumi-3 (CD33+/CD7+) cells per well. The plates were incubated at 37° C., 5% CO 2 for 96 hours. Following incubation, 5 μl of Alamar Blue reagent was added per well and the plates read following a further incubation at 37° C., 5% CO₂ for 4 and 6 hours. The data for each reading was plotted in GraphPad PRISM and the IC50 values recorded. The data from the 4-hour read for cell line Kasumi-3 was used to calculate the 1050. The IC50 was 0.1235 nM;

FIG. 4 is a graph describing a cell kill assay conducted using a 10-point dose response of directly conjugated BVX130-MMAF on 2×10⁴ HNT-34 (CD33+/CD7+) cells per well. The plates were incubated at 37° C., 5% CO 2 for 96 hours. Following incubation, 5 μl of Alamar Blue reagent was added per well and the plates read following a further incubation at 37° C., 5% CO₂ for 4 and 6 hours. The data for each reading was plotted in GraphPad PRISM and the IC50 values recorded. The data from the 4-hour read for cell line HNT-34s was used to calculate the 1050. 1050 was 0.1204 nM;

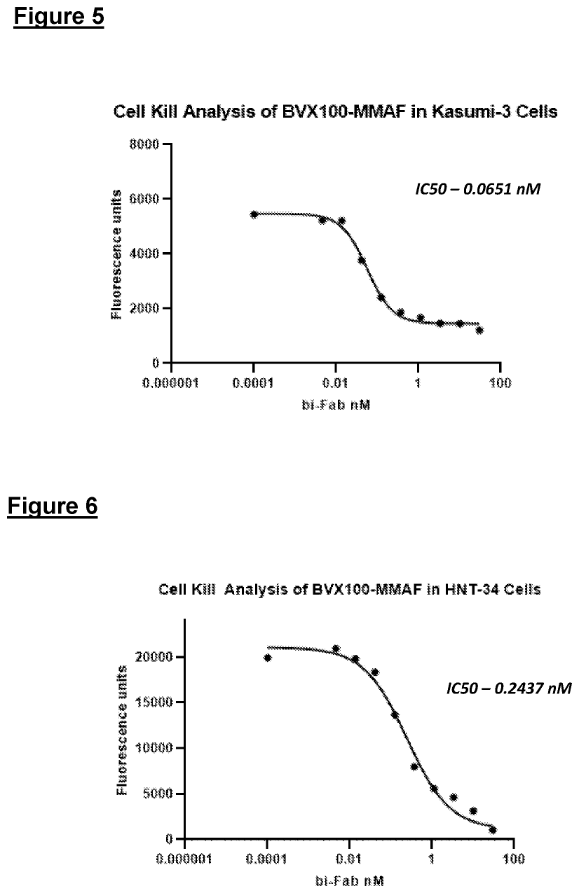

FIG. 5 is a graph describing a cell kill assay conducted using a 10-point dose response of directly conjugated BVX100-MMAF on 2×10⁴ Kasumi-3 (CD33+/CD7+) cells per well. The plates were incubated at 37° C., 5% CO₂ for 96 hours. Following incubation, 5 μl of Alamar Blue reagent was added per well and the plates read following a further incubation at 37° C., 5% CO₂ for 4 and 6 hours. The data for each reading was plotted in GraphPad PRISM and the IC50 values recorded. The data from the 4-hour read for cell line Kasumi-3 was used to calculate the 1050. The IC50 was 0.0651 nM;

FIG. 6 is a graph describing a cell kill assay conducted using a 10-point dose response of directly conjugated BVX100-MMAF on 2×10⁴ HNT-34 (CD33+/CD7+) cells per well. The plates were incubated at 37° C., 5% CO₂ for 96 hours. Following incubation, 5 μl of Alamar Blue reagent was added per well and the plates read following a further incubation at 37° C., 5% CO₂ for 4 and 6 hours. The data for each reading was plotted in GraphPad PRISM and the IC50 values recorded. The data from the 4-hour read for cell line HNT-34 was used to calculate the 1050. The IC50 was 0.2437 nM;

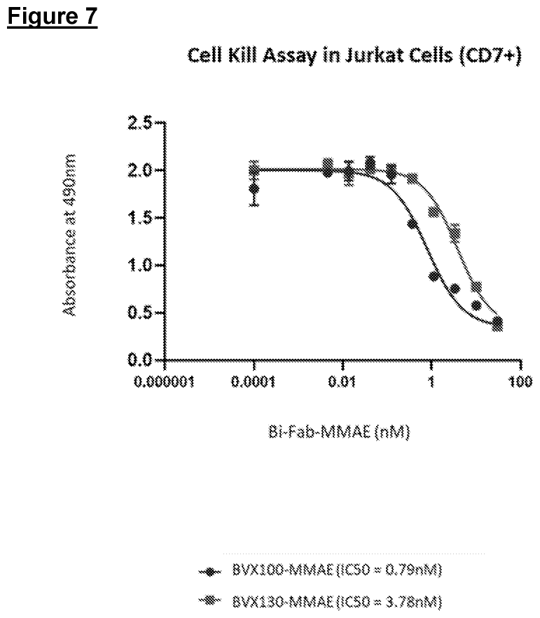

FIG. 7 is a graph that shows the dose response curve of a cell kill assay conducted using a 9-point dose of directly conjugated BVX130-MMAE or BVX100-MMAE on 2×10⁴ Jurkat cells (CD33−/CD7+) per well, to a top final concentration of 30 nM. The plates were incubated at 37° C., 5% CO2 for 96 hours. Following incubation, 10 μl of CellTiter 96 AQueous One Solution was pipetted per well and the plates incubated at 37° C., 5% CO2 for a further 3 hours. The absorbance was read at 492 and 690 nm. The OD 690 nm was subtracted from the OD 492 nm and the data plotted using GraphPad PRISM software. Error bars represent the standard deviation of quadruplicate repeats;

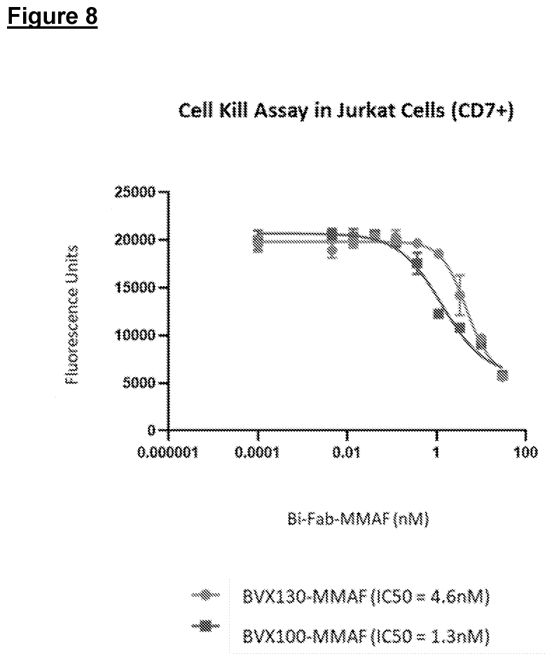

FIG. 8 is a graph that shows the dose response curve of a cell kill assay conducted using a 10-point dose response of directly conjugated BVX130-MMAF or BVX100-MMAF on 2×10⁴ Jurkat cells (CD33−/CD7+) per well, to a top final concentration of 30 nM. The plates were incubated at 37° C., 5% CO2 for 96 hours. Following incubation, 5 μl of Alamar Blue reagent was added per well and the plates read following a further incubation at 37° C., 5% CO2 for 4 and 6 hours. The data for each reading was plotted in GraphPad PRISM and the IC50 values recorded. The data from the 4-hour read for the Jurkat cell line was used to calculate the IC50. Error bars represent the standard deviation of quadruplicate repeats; and

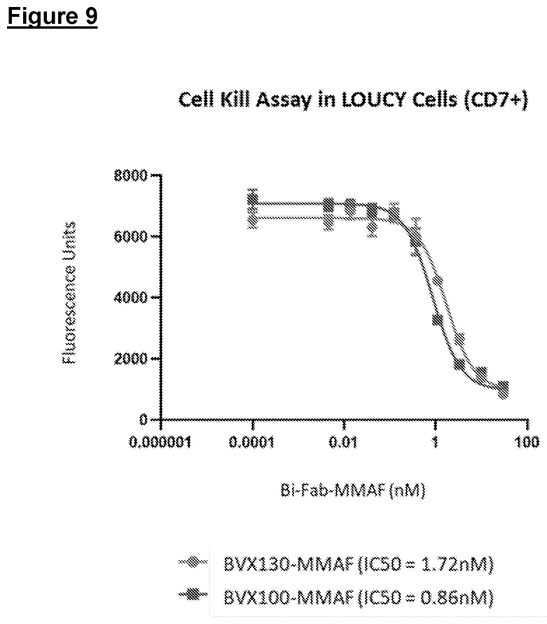

FIG. 9 is a graph that shows the dose response curve of a cell kill assay conducted using a 10-point dose response of directly conjugated BVX130-MMAF or BVX100-MMAF on 2×10⁴ LOUCY cells (CD33−/CD7+) per well, to a top final concentration of 30 nM. The plates were incubated at 37° C., 5% CO2 for 96 hours. Following incubation, 5 μl of Alamar Blue reagent was added per well and the plates read following a further incubation at 37° C., 5% CO2 for 4 and 6 hours. The data for each reading was plotted in GraphPad PRISM and the IC50 values recorded. The data from the 4-hour read for the LOUCY cell line was used to calculate the IC50. Error bars represent the standard deviation of quadruplicate repeats.

### Example 1—Assessing the Binding of Different CD33+/CD7+ bi-Fabs in Cell Lines and Primary Cells

The binding of different CD33+/CD7+ conjugated bispecific antibodies was assessed in a range of cell lines and the primary cells derived from healthy human donors. Each of the conjugated bispecific antibodies demonstrated a different pair of binding arms that were targeted towards either CD33 or CD7 (table 1). Each of the binding arms of the bispecific antibodies demonstrate different affinities for either CD33 or CD7.

TABLE 1
Bispecific antibodies assessed for binding
to a range of cellular test system.

| | Construct # | | |
|---|---|---|---|
| Candidate # | CD7 | CD33 | Mutant |
| BVX0011/BVX1001 | 0 | 0 | CD33-PP-1 WT/CD7-PP-1 WT |
| BVX1101 | 1 | 0 | CD33-PP-1 WT/CD7-PP-2 |
| BVX1201 | 2 | 0 | CD33-PP-1 WT/CD7-PP-3 |
| BVX1301 | 3 | 0 | CD33-PP-1 WT/CD7-PP-6 |
| BVX1401 | 4 | 0 | CD33-PP-1 WT/CD7-PP-7 |
| BVX1501 | 5 | 0 | CD33-PP-1 WT/CD7-PP-8 |
| BVX1601 | 6 | 0 | CD33-PP-1 WT/CD7-PP-13 |
| BVX1011 | 0 | 1 | CD33-PP-4/CD7-PP-1 WT |
| BVX1611 | 6 | 1 | CD33-PP-4/CD7-PP-13 |
| BVX1021 | 0 | 2 | CD33-PP-7/CD7-PP-1 WT |
| BVX1521 | 5 | 2 | CD33-PP-7/CD7-PP-8 |
| BVX1621 | 6 | 2 | CD33-PP-7/CD7-PP-13 |
| BVX1631 | 6 | 3 | CD33-PP-11/CD7-PP-13 |

TABLE 2
Nomenclature used to differentiate and identify each bispecific
antibody. For example, BVX1301 corresponds to programme number
1, CD7 construct 3, CD33 construct WT, and MMAE payload.
BVX1302 would replace the MMAE payload with MMAF.
Nomenclature

| | | | | | |
|---|---|---|---|---|---|
| BVX | Programme # | CD7 | CD33 | Dash | Payload # |
| | | Construct # | Construct # | (—) | |

### Reagents

| | |
|---|---|
| Mouse anti-Human IgG Fab Secondary | ThermoFisher #MA110377 |
| Antibody, PE | |
| Jurkat Cells | DSMZ 282 |
| Kasumi-3 Cells | DSMZ 714 |
| HNT-34 Cells | DSMZ 600 |
| SHI-1 Cells | DSMZ 645 |
| Healthy PBMCs Donor 1 | Cambridge Bioscience ID |
| PR18E125592 | |
| PBS/A (Cat #. 50086470) | VWR |
| BSA (Bovine serum albumin | ThermoFisher |
| Fract V 100 ml 7.5% Cat #. 15260037) | |
| BVX1001 | |
| BVX1101 | |
| BVX1201 | |
| BVX1301 | |
| BVX1401 | |
| BVX1501 | |
| BVX1601 | |
| BVX1011 | |
| BVX1611 | |
| BVX1021 | |
| BVX1521 | |
| BVX1621 | |
| BVX1631 | |

### Method

1 ml of each bi-Fab agent was prepared at 10 nM in PBS/0.1% BSA. The cells were harvested and counted so there were enough cells to test each bi-Fab with each bi-Fab dilution, plus a sample labelled with secondary antibody only, using 50,000 cells per test. For the PBMC sample 100,000 cells were used per test. The cells were pelleted at 1000 rpm for 5 minutes at 4° C. and resuspended in ice cold PBS. 100 μl aliquots of each cell sample was pipetted across wells of a V-shape bottomed 96 well plate on ice. The cells were pelleted at 1000 rpm for 5 minutes at 4° C. and the supernatant aspirated. The cells were resuspended in 100 μl of 10 nM bi-Fab. The plates were incubated on ice for 1 hour. 75 μl of ice-cold PBS was added to each well and the cells pelleted at 1000 rpm for 5 minutes at 4° C. The supernatant was aspirated and the cells resuspended in 50 μl of Mouse anti-Human IgG Fab Secondary Antibody, PE diluted 1 in 17 in ice cold PBS/0.1% BSA. The plates were incubated on ice for 1 hour. 100 μl of ice-cold PBS was added per well and the cells pelleted at 1000 rpm for 5 minutes at 4° C. The cells were resuspended in 300 μl of ice-cold PBS, transferred to FACS tube and fluorescence analysed using the FACS Calibur, detecting PE staining in FL2.

The monocyte populations in the PBMC sample were identified from the Side scatter/Forward scatter dot plot. The data was plotted in excel.

### Results

The binding of each bispecific antibody (1 nM and 10 nM) to each cell line was assessed alongside the binding to monocytes and T-cells isolated from healthy human donors (FIG. 1 and FIG. 2). It is clear from the data that the specific CD7 and CD33 constructs used within each bispecific construct has an impact on the binding to double (CD33+/CD7+) vs single (CD33+/CD7− or CD33−/CD7+) antigen positive cell lines and primary cells isolated from healthy human donors.

TABLE 2
Bispecific constructs

| | | | | | | | | | Fold | Fold |
|---|---|---|---|---|---|---|---|---|---|---|
| | | | | | | | | FACS | re- | Re- |
| | | | | | | | | fold | duction | duction |
| | | | | | | | | re- | in | in |
| | | | | | Affinity | | | duction | binding | binding |
| | | | | Final | from | | | com- | affinity | affinity |
| | | | Position | Yeild | cell | Affinity | | pared | com- | com- |
| | Single/ | Position of | of | Post | capt. | from | | to | pared | pared |
| | Double | Mutation | Mutation | Purifi- | exp. | SPR | FACS | PP | to WT | to PP |
| Mutant # | Mutant | #1 | #2 | cation | (μγ/ml) | (Kd) | binding | WT | (Kd | WT (Kd) |
| 7-AA-WT | WT | | | 1 mg | 23 | 2.63E−09 | | | | |
| 7-AA-1 | Single | Heavy Chain CDR3 | | 1 mg | 46 | 4.45E−08 | | | 17 | |
| | | Tyr108Ala | | | | | | | | |
| 7-AA-2 | Single | Heavy Chain CDR3 | | 1 mg | NO | 3.44E−06 | | | 1310 | |
| | | Tyr107Ala | | | BINDING | | | | | |
| 7-AA-3 | Single | Heavy Chain CDR3 | | 0.5 mg | NO | 1.92E−07 | | | 73 | |
| | | Tyr106Ala | | | BINDING | | | | | |
| 7-AA-4 | Single | Heavy Chain CDR3 | | Failure | NA | NA | | | | |
| | | Tyr103Ala | | | | | | | | |
| 7-AA-5 | Single | Light Chain CDR3 | | Failure | NA | NA | | | | |
| | | Trp94Ala | | | | | | | | |
| 7-AA-6 | Single | Light Chain CDR3 | | Failure | NA | NA | | | | |
| | | Trp94Leu | | | | | | | | |
| 7-AA-7 | Double | Heavy Chain CDR3 | Light | Failure | NA | NA | | | | |
| | | Tyr108Ala | Chain | | | | | | | |
| | | | CDR3 | | | | | | | |
| | | | Trp94Ala | | | | | | | |
| 7-AA-8 | Double | Heavy Chain CDR3 | Light | Failure | NA | NA | | | | |
| | | Tyr107Ala | Chain | | | | | | | |
| | | | CDR3 | | | | | | | |
| | | | Trp94Ala | | | | | | | |
| 7-AA-9 | Double | Heavy Chain CDR3 | Light | Failure | NA | NA | | | | |
| | | Tyr106Ala | Chain | | | | | | | |
| | | | CDR3 | | | | | | | |
| | | | Trp94Ala | | | | | | | |
| 7-AA-10 | Double | Heavy Chain CDR3 | Light | Failure | NA | NA | | | | |
| | | Tyr103Ala | Chain | | | | | | | |
| | | | CDR3 | | | | | | | |
| | | | Trp94Ala | | | | | | | |
| 7-AA-11 | Double | Heavy Chain CDR3 | Light | Failure | NA | NA | | | | |
| | | Tyr108Ala | Chain | | | | | | | |
| | | | CDR3 | | | | | | | |
| | | | Trp94Leu | | | | | | | |
| 7-AA-12 | Double | Heavy Chain CDR3 | Light | Failure | NA | NA | | | | |
| | | Tyr107Ala | Chain | | | | | | | |
| | | | CDR3 | | | | | | | |
| | | | Trp94Leu | | | | | | | |
| 7-AA-13 | Double | Heavy Chain CDR3 | Light | Failure | NA | NA | | | | |
| | | Tyr106Ala | Chain | | | | | | | |
| | | | CDR3 | | | | | | | |
| | | | Trp94Leu | | | | | | | |
| 7-AA-14 | Double | Heavy Chain CDR3 | Light | Failure | NA | NA | | | | |
| | | Tyr103Ala | Chain | | | | | | | |
| | | | CDR3 | | | | | | | |
| | | | Trp94Leu | | | | | | | |
| 7-AA-15 | Double | Heavy Chain CDR1 | Light | Not started | NA | NA | | | | |
| | | Tyr32Ala | Chain | | | | | | | |
| | | | CDR3 | | | | | | | |
| | | | Trp94Leu | | | | | | | |
| 7-AA-16 | Double | Heavy Chain CDR2 | Light | Not started | NA | NA | | | | |
| | | Asn52Ala | Chain | | | | | | | |
| | | | CDR3 | | | | | | | |
| | | | Trp94Leu | | | | | | | |
| 7-PP-2 | Single | Heavy Chain | | 32.7 mg | | 7.57E−08 | 34.60 | 64.92 | 28.84 | 21.26 |
| | | Arg57Gly | | | | | | | | |
| 7-PP-3 | Single | Heavy Chain | | 84.6 mg | | 3.25E−08 | 13.04 | 24.47 | 12.38 | 9.13 |
| | | Arg57Lys | | | | | | | | |
| 7-PP-4 | Single | Heavy Chain | | 55.4 mg | | — | 1992.00 | 3737.34 | | |
| | | Tyr102Thr | | | | | | | | |
| 7-PP-5 | Single | Heavy Chain | | 80.2 mg | | — | 268.30 | 503.38 | | |
| | | Leu 105Gly | | | | | | | | |
| 7-PP-6 | Single | Heavy Chain | | 72.3 mg | | 4.52E−08 | 9.76 | 18.32 | 17.22 | 12.70 |
| | | Asp104Ala | | | | | | | | |
| 7-PP-7 | Double | Heavy Chain | | 6.2 mg | | 5.41E−07 | 267.30 | 501.50 | 206.10 | 151.97 |
| | | Arg57Gly/ | | | | | | | | |
| | | Tyr108Ala | | | | | | | | |
| 7-PP-8 | Double | Heavy Chain | | 63.4 mg | | 2.29E−07 | 96.75 | 181.52 | 87.24 | 64.33 |
| | | Arg57Lys/ | | | | | | | | |
| | | Tyr108Ala | | | | | | | | |
| 7-PP-9 | Double | Heavy Chain | | 78.8 mg | | — | 275.30 | 516.51 | | |
| | | Tyr102Thr/ | | | | | | | | |
| | | Tyr108Ala | | | | | | | | |
| 7-PP-10 | Double | Heavy Chain | | 88.0 mg | | — | 334.90 | 628.33 | | |
| | | Leu106Gly/ | | | | | | | | |
| | | Tyr108Ala | | | | | | | | |
| 7-PP-11 | Double | Heavy Chain | | 57.7 mg | | 2.46E−07 | 55.85 | 104.78 | 93.71 | 69.10 |
| | | Asp104Ala/ | | | | | | | | |
| | | Tyr108Ala | | | | | | | | |
| 7-PP-12 | Single | | Light | 71.0 mg | | 5.46E−09 | 0.002 | 0.004 | 2.08 | 1.53 |
| | | | Chain | | | | | | | |
| | | | Gln27Ala | | | | | | | |
| 7-PP-1 | | | | 21.8 mg | | 3.56E−09 | 0.53 | 1.00 | 1.36 | |
| WT | | | | | | | | | | |
| 7-PP-13 | | Heavy Chain CDR3 | | 50.6 mg | | 7.22E−08 | 28.38 | 53.25 | 27.50 | 20.28 |
| (mut_1 7- | | Tyr108Ala | | | | | | | | |
| AA-1) | | | | | | | | | | |
| A. | | | | | | | | | | |

| | | | | | | Fold |
|---|---|---|---|---|---|---|
| | | | | | | Reduction |
| | | | | | | in binding |
| | | | | | | affinity |
| | | Position | Position | Affinity | | compared |
| | Single/ | of | of | from | FACS | to 33- |
| | Double | Mutation | Mutation | SPR | binding | PP-1 WT |
| Mutant # | Mutant | #1 | #2 | (Kd) | (nM) | (Kd, nM) |
| 33-AA- | Wildtype | | | 2.96E−09 | | |
| WT | (WT) | | | | | |
| 33-AA-1 | Single | Heavy Chain CDR1 | | 2.62E−09 | | |
| 33-AA-2 | Single | Heavy Chain CDR2 | | NA | | |
| 33-AA-3 | Single | Heavy Chain CDR2 | | 3.49E−09 | | |
| 33-AA-4 | Single | Heavy Chain CDR3 | | NA | | |
| 33-AA-5 | Single | Heavy Chain CDR3 | | 8.82E−09 | | |
| 33-AA-6 | Single | Light Chain CDR1 | | NA | | |
| 33-AA-7 | Single | Light Chain CDR3 | | 8.68E−09 | | |
| 33-AA-8 | Double | Heavy Chain CDR2 | Light Chain | 7.89E−07 | | |
| | | | CDR3 | | | |
| 33-AA-9 | Double | Heavy Chain CDR2 | Light Chain | 9.24E−09 | | |
| | | | CDR3 | | | |
| 33-AA-10 | Double | Heavy Chain CDR3 | Light Chain | NA | | |
| | | | CDR3 | | | |
| 33-AA-11 | Double | Heavy Chain CDR3 | Light Chain | 1.19E−08 | | |
| | | | CDR3 | | | |
| 33-AA-12 | Double | Heavy Chain CDR2 | Light Chain | NA | | |
| | | | CDR1 | | | |
| 33-AA-13 | Double | Heavy Chain CDR2 | Light Chain | NA | | |
| | | | CDR1 | | | |
| 33-AA-14 | Double | Heavy Chain CDR3 | Light Chain | NA | | |
| | | | CDR1 | | | |
| 33-PP-2 | Single | | Light Chain | 1.28E−09 | 0.16 | 0.94 |
| 33-PP-3 | Single | Heavy Chain | | 1.95E−09 | 0.19 | 1.43 |
| 33-PP-4 | Single | Heavy Chain | | 5.27E−08 | 321.10 | 38.75 |
| 33-PP-5 | Double | Heavy Chain | Light Chain | 1.75E−09 | 0.18 | 1.29 |
| 33-PP-6 | Double | Heavy Chain | | 1.72E−09 | 0.15 | 1.26 |
| 33-PP-7 | Double | Heavy Chain | | 9.23E−08 | 464.50 | 67.87 |
| 33-PP-8 | Single | | Light Chain | 2.01E−07 | 686.5 | 147.79 |
| 33-PP-9 | Double | Heavy Chain | Light Chain | 3.07E−07 | 1245.00 | 225.74 |
| 33-PP-10 | Double | Heavy Chain | Light Chain | 2.72E−06 | 147818.00 | 2000.00 |
| 33-PP-1 | | | | 1.36E−09 | 0.16 | |
| WT | | | | | | |
| B. | | | | | | |

A. CD7 constructs;
B. CD33 constructs

FIGS. 1 and 2 show that the reduction in binding affinity of the CD7 binding arm of a bispecific antibody that also contains the wild type CD33 binding arm does not affect the binding affinity to CD7+/CD33+ cell lines. In a surprising finding, the binding of the bispecific constructs to CD33-/CD7+ cells were affected by the affinity of both the CD7 and the CD33 arm used within the construct. Table 2 above shows the results of the bispecific antibodies tested and this data is summarised in Table 3 which includes the fold reduction in binding affinity of each binding arm of each bispecific antibody.

TABLE 3
Overview of the binding affinity (Kd) of each bispecific antibody
candidate compared to wildtype as measured by SPR

| | | | Fold reduction | Fold reduction |
|---|---|---|---|---|
| | | | in binding | in binding |
| Construct | CD7 | CD33 | affinity | affinity |
| Name | construct | construct | vs 7-PP-1 WT | vs 33-PP-1 WT |
| BVX100 | 7-PP-1 WT | 33-PP-1 WT | 0 | 0 |
| BVX110 | 7-PP-2 | 33-PP-1 WT | 21.3 | 0 |
| BVX120 | 7-PP-3 | 33-PP-1 WT | 9.1 | 0 |
| BVX130 | 7-PP-6 | 33-PP-1 WT | 12.7 | 0 |
| BVX140 | 7-PP-7 | 33-PP-1 WT | 152 | 0 |
| BVX150 | 7-PP-8 | 33-PP-1 WT | 64.3 | 0 |
| BVX160 | 7-PP-13 | 33-PP-1 WT | 20.28 | 0 |
| BVX101 | 7-PP-1 WT | 33-PP-4 | 0 | 38.8 |
| BVX102 | 7-PP-1 WT | 33-PP-7 | 0 | 68 |
| BVX152 | 7-PP-8 | 33-PP-7 | 87 | 68 |
| BVX161 | 7-PP-13 | 33-PP-4 | 20.28 | 39 |
| BVX162 | 7-PP-13 | 33-PP-7 | 20.28 | 68 |

### Example 2—Assessing Cytotoxicity in CD7+CD33+ Immortalised Cell Lines

The cytotoxicity (cell kill) efficiency of CD33+/CD7+ bispecific antibody drug conjugates was assessed in multiple CD33+/CD7+ cell lines.

### Test Agents

BVX100-MMAF—CD33 monobinder×CD7 monobinder bi-Fab conjugated to mcMMAF (cytotoxic payload)
BVX130-MMAF—CD33 monobinder×CD7 monobinder bi-Fab conjugated to mcMMAF (cytotoxic payload) with partially optimised CD7 monobinder (i.e. different CD7 protein sequence compared to BVX100-MMAF)
CD33 x CD33-MMAF—CD33 monobinder×CD33 monobinder bi-Fab conjugated to mcMMAF (cytotoxic payload)
Gemtuzumab-MMAF—Gemtuzumab is the commercial CD33 monospecific IgG antibody component of Pfizer's ADC Mylotarg™, conjugated to mcMMAF (cytotoxic payload)
All ADC conjugates used the same payload, conjugation technology and same Drug:Antibody Ratio

### Reagents

| | |
|---|---|
| Kasumi-3 Cells | DSMZ 714 |
| HNT-34 Cells | DSMZ 600 |
| BVX130-MMAF (P01-32, 10.5 uM) | In House - Head of Chemistry |
| BVX130-MMAE (193-27-3, 49 uM) | In House - Head of Chemistry |
| BVX100-MMAF (193-15, 13 uM) | In House - Head of Chemistry |
| BVX100-MMAE (193-28, 34 uM) | In House - Head of Chemistry |
| BSA (Bovine serum albumin Fract V | ThermoFisher |
| 100 ml 7.5% Cat #. 15260037) | |
| PBS/A (Cat #. 50086470) | VWR |
| Corning 384 Well Clear Flat Bottom | SLS |
| Polystyrene TC-Treated Microplates | |
| alamarBlue ™ Cell Viability Reagent | ThermoFisher |
| (DAL1025) | |
| RPMI-1640 medium 21875034 | Gibco, Life Technologies |
| GlutaMAX ™ Supplement 35050061 | Gibco, Life Technologies |
| Fetal Bovine Serum, | Gibco, Life Technologies |
| Heat inactivated 10500064 | |
| Penicillin-Streptomycin | Gibco, Life Technologies |
| (10,000 U/mL) 15140122 | |

### Method

Kasumi-3 and HNT-34 cell lines were harvested, counted and the volume required to seed 2000 cells per well across 100 wells in 40 μl media per well was calculated for a 384-well plate. A 10-point dose response of BVX130-MMAE, BVX130-MMAF, BVX100-MMAE and BVX100-MMAF were prepared in Assay Media (RPMI, 10% FBS, 1% Glutamax, 1% Pen/Strep) at 5× the final concentration with a top final concentration of 30 nM. 10 μl of each dose was pipetted across triplicate wells in a 384 well plate and a separate plate was prepared for each cell line tested. 500,000 cells from each cell line were pipetted into 5 ml of Assay Media and the cells pelleted. Each cell pellet was resuspended in 10 mls of Assay Media and 40 μl pipetted into the indicated wells. 50 μl of Assay Media was pipetted in the Blank control wells and 50 μl of PBS was pipetted into all the spare wells and the plates incubated at 37° C., 5% CO₂ for 4 or 7 days. Following 4 days incubation 5 μl of Alamar Blue reagent was added per well and the plates read following a further incubation at 37° C., 5% CO₂ for 4 and 6 hours. The data for each reading was plotted in GraphPad PRISM and the IC50 values recorded. The data from the 4-hour read for cell lines Kasumi-3 and HNT-34 was used in the IC50 summary table.

### Results

Example dose response curves for each of these assays are presented in FIGS. 3 to 6. Briefly, each of the CD33+/CD7+ bispecific antibody drug conjugates showed robust cytotoxicity in different CD33+/CD7+ cell lines. Table 4 below shows an overview of the average cytotoxicity IC50 values for each bispecific antibody in each cell type across multiple experiments.

TABLE 4
Mean IC50's for CD33+CD7+ cell lines Kasumi-3
and HNT-34 across multiple experiments with CD7/CD33
bispecific antibody drug conjugates

| | Kasumi-3 | HNT-34 |
|---|---|---|
| Mean IC50 when treated with | 0.16 | 0.37 |
| BVX130-MMAF (nM) | | |
| Number of Experiments (n=) | 13 | 12 |
| Mean IC50 when treated with | 0.45 | 0.40 |
| BVX130-MMAE (nM) | | |
| Number of Experiments (n=) | 6 | 8 |
| Mean IC50 when treated with | 0.14 | 0.19 |
| BVX100-MMAE (nM) | | |
| Number of Experiments (n=) | 9 | 8 |
| Mean IC50 when treated with | 0.11 | 0.41 |
| BVX100-MMAF (nM) | | |
| Number of Experiments (n=) | 6 | 4 |

FIG. 3 is a graph that shows the cytotoxicity dose response curve produced when Kasumi-3 cells are exposed to increasing concentrations of BVX130-MMAF. Kasumi-3 cells are CD33+/CD7+ and BVX130-MMAF is an affinity reduced CD33+/CD7+ bispecific antibody conjugated to a cytotoxic payload. The example IC50 of BVX130-MMAF in Kasumi-3 cells is 0.1235 nM and the average IC50 across thirteen experiments is 0.16 nM. The average IC50 of the same bispecific antibody conjugate in HNT-34 cells (CD33+/CD7+) across twelve experiments is 0.37 nM. FIG. 4 is an example dose response curve that is produced when HNT-34s cells are exposed to increasing concentrations of BVX130-MMAF.

BVX130-MMAF comprises a partially optimised CD7 sequence compared with BVX100-MMAF. FIGS. 5 and 6 are example dose response curves produced using BVX100-MMAF as the conjugated bispecific antibody drug conjugate. As with BVX130-MMAF the cell types tested were Kasumi-3 and HNT-34; both are CD33+/CD7+. The average 1050 of BVX100-MMAF in Kasumi-3 cells is 0.11 nM across six experiments. The average IC50 of BVX100-MMAF in HNT-34 cells is 0.41 nM across four experiments.

The MMAF conjugated bispecific antibodies generally demonstrate a higher IC50 in each cell type as shown in Table 4. For example, an average IC50 of 0.16 nM for BVX130-MMAF in Kasumi-3 cells versus an IC50 of 0.45 nM for BVX130-MMAE in the same cell type. The same is true when considering BVX100-MMAF vs BVX100-MMAE in the same cell line.

### SUMMARY

The results described above demonstrate that each of the bispecific CD33+/CD7+ antibodies conjugated to a cytotoxic payload cause effective cell death in two CD33+/CD7+ cells lines. The next step was to determine whether exposure to these bispecific antibody drug conjugates would cause off-target cytotoxicity in cells that express just CD33 or CD7 as single targets, representing single antigen positive healthy cell populations in the blood. This was assessed by determining fold selectivity in cell kill in CD33+/CD7+ cell lines vs CD33+/CD7− and CD33−/CD7+ cell lines using the bispecific antibody panel conjugated to a cytotoxic payload.

### Example 3—Assessing Selectivity for CD33+/CD7+ Over CD33+/CD7− and CD33−/CD7+ Cells

In an effort to determine whether certain bispecific antibodies conjugated to cytotoxic payloads preferentially targeted CD33+/CD7+ cells over CD33+/CD7− and CD33−/CD7+ cells, a panel of double and single antigen positive immortalised cell lines were exposed to a range of conjugated CD7+/CD33+ bispecific antibodies and the cytotoxicity measured.

### Cytotoxic Activity of Bi-Fab-MMAE Conjugates in CD33 and CD7 Double and Single Positive Cell Lines

### Reagents

| | |
|---|---|
| Kasumi-3 cells | DSMZ 714 |
| HNT-34 cells | DSMZ 600 |
| SHI-1 cells | DSMZ 645 |
| MV4.11 cells | DSMZ 102 |
| Jurkat Cells | DSMZ 282 |
| BVX100-MMAE | In House - Head of Chemistry |
| BVX110-MMAE | |
| BVX120-MMAE | |
| BVX130-MMAE | |
| BVX140-MMAE | |
| BVX150-MMAE | |
| BVX160-MMAE | |
| BSA (Bovine serum albumin Fract V | ThermoFisher |
| 100 ml 7.5% Cat #. 15260037) | |
| PBS/A (Cat #. 50086470) | VWR |
| Corning 384 Well Clear Flat Bottom | SLS |
| Polystyrene TC-Treated Microplates | |
| alamarBlue ™ Cell Viability Reagent | ThermoFisher |
| (DAL1025) | |
| RPMI-1640 medium 21875034 | Gibco, Life Technologies |
| GlutaMAX ™ Supplement 35050061 | Gibco, Life Technologies |
| Fetal Bovine Serum, | Gibco, Life Technologies |
| Heat inactivated 10500064 | |
| Penicillin-Streptomycin | Gibco, Life Technologies |
| (10,000 U/mL) 15140122 | |

### Method

Cells were harvested, counted and resuspended at 2×10⁴ cells per 90 μl growth media. An 8-point dose response of the bi-Fab-MMAE was prepared in growth media at 5 times the final assay concentration, with a final top concentration of 30 nM. 10 μl of bi-Fab-MMAE titration was pipetted into a 384-well plate and each concentration was tested across duplicate wells. 40 μl of cells were pipetted into the wells and the plates incubated at 37° C., 5% CO2 for 96 hours. Following incubation, 5 μl of MTS reagent was pipetted per well and the plates incubated at 37° C., 5% CO2 for a further 3 hours. The absorbance was read at 492 and 690 nm. The OD 690 nm was subtracted from the OD 492 nm and the data plotted using GraphPad PRISM software.

TABLE 5
Cytotoxic Activity of CD33+/CD7+ Bispecific-MMAE Conjugates in
CD33 and CD7 Double and Single Positive Cell Lines

| | | | | | | | | Fold | Fold |
|---|---|---|---|---|---|---|---|---|---|
| | | | | | | | | Selec- | Selec- |
| | | | | | | | | tivity | tivity |
| | | | | | | | | of | of |
| | | | EC₅₀ | | | | | CD7⁺/ | CD7⁺/ |
| | EC₅₀ (nM) | | (nM) | EC₅₀ (nM) | | EC₅₀ (nM) | | CD33⁺ | CD33⁺ |
| Bi- | CD7⁺/CD33⁺ | | CD7⁺/ | CD7⁻/CD33⁺ | | CD7⁻/CD33⁻ | | vs | vs |
| Fab- | HNT- | Kasumi- | CD33⁻ | MV4. | SHI- | | DND- | CD7⁺/ | CD7⁻/ |
| MMAE | 34 | 3 | Jurkat | 11 | 1 | Ramos | 39 | CD33⁻ | CD33⁺ |
| BVX100 | 0.19 | 0.14 | 0.92 | 10.77 | 2.26 | NR | NR | 5.4 | 38 |
| | (+0.04) | (+0.07) | (+0.22) | (+3.69) | (+0.89) | | | | |
| BVX110 | 0.61 | 0.82 | 4.52 | 26.97 | 11.39 | NR | NR | 6.5 | 27.4 |
| | (+0.01) | (+0.38) | (+1.16) | (+1.51) | (+0.99) | | | | |
| BVX120 | 0.23 | 0.26 | 1.52 | 16.52 | 7.21 | NR | NR | 6.1 | 47.5 |
| | (+0.02) | (+0.11) | (+0.34) | (+0.86) | (+0.91) | | | | |
| BVX130 | 0.4 | 0.45 | 5.83 | 24.07 | 17.48 | NR | NR | 13.6 | 48.3 |
| | (+0.19) | (+0.07) | (+1.25) | (+3.25) | (+3.28) | | | | |
| BVX140 | 17.23 | 24.91 | 22.80 | NR | NR | NR | NR | 1.1 | >1.4 |
| | (+4.28) | (+3.94) | (+5.0) | | | | | | |
| BVX150 | 0.63 | 0.76 | 1.60 | 9.19 | 5.23 | NR | NR | 2.3 | 10.3 |
| | (+0.17) | (+0.20) | (+0.54) | (+0.17) | (+0.29) | | | | |
| BVX160 | 2.22 | 1.32 | 7.31 | NR | NR | NR | NR | 4.2 | >17 |
| | (+0.24) | (+0.14) | (+2.14) | | | | | | |

### Results

The determined cytotoxicity for each conjugated bispecific antibody tested across the full cell line panel is given in Table 5, along with the fold selectivity of each construct for double positive CD33+/CD7+ cell lines compared to either CD33+/CD7− or CD33−/CD7+ cell lines. From these results, BVX130 was selected for further analysis due to the potent cytotoxicity seen in CD33+/CD7+ cell lines and the increased fold selectivity seen vs CD33+/CD7− and CD33−/CD7+ cell lines.

### Cytotoxic Activity of BVX100 and BVX130 MMAF Conjugates in CD7 Single Positive Cell Lines

### Reagents

| Jurkat Cells | DSMZ 282 |
|---|---|
| LOUCY Cells | DSMZ 394 |
| BVX130-MMAF (P01-32, 10.5 uM) | In House - Head of Chemistry |
| BVX100-MMAF (193-15, 13 uM) | In House - Head of Chemistry |
| BSA (Bovine serum albumin | ThermoFisher |
| Fract V 100 ml 7.5% Cat #. 15260037) | |
| PBS/A (Cat #. 50086470) | VWR |
| Corning 384 Well Clear Flat Bottom | |
| Polystyrene TC-Treated Microplates | SLS |
| alamarBlue ™ Cell Viability Reagent | ThermoFisher |
| (DAL1025) | |
| RPMI-1640 medium 21875034 | Gibco, Life Technologies |
| GlutaMAX ™ Supplement 35050061 | Gibco, Life Technologies |
| Fetal Bovine Serum, Heat inactivated | Gibco, Life Technologies |
| 10500064 | |
| Penicillin-Streptomycin (10,000 U/mL) | Gibco, Life Technologies |
| 15140122 | |

### Method

Jurkat and LOUCY cell lines were harvested, counted and the volume required to seed 2000 cells per well across 100 wells in 40 μl media per well was calculated for a 384-well plate. A 10-point dose response of BVX130-MMAF and BVX100-MMAF were prepared in Assay Media (RPMI, 10% FBS, 1% Glutamax, 1% Pen/Strep) at 5× the final concentration with a top final concentration of 30 nM. 10 μl of each dose was pipetted across triplicate wells in a 384 well plate and a separate plate was prepared for each cell line tested. 500,000 cells from each cell line were pipetted into 5 ml of Assay Media and the cells pelleted. Each cell pellet was resuspended in 10 mls of Assay Media and 40 μl pipetted into the indicated wells. 50 μl of Assay Media was pipetted in the Blank control wells and 50 μl of PBS was pipetted into all the spare wells and the plates incubated at 37° C., 5% CO2 for 4 days. Following 4 days incubation 5 μl of Alamar Blue reagent was added per well and the plates read following a further incubation at 37° C., 5% CO2 for 4 and 6 hours. The data for each reading was plotted in GraphPad PRISM and the IC50 values recorded.

### Results

The affinity modulated CD33+/CD7+ bispecific antibody BVX130 conjugated to either MMAF or MMAE cytotoxic payloads demonstrate reduced cytotoxicity in CD7 single antigen positive cells compared to BVX100, as can be seen in FIGS. 7 to 9.

As has been demonstrated, CD33+/CD7+ bispecific antibodies conjugated to a cytotoxic payload cause robust cytotoxicity in immortalised CD33+/CD7+ cell lines (FIGS. 3 to 6). Reduced cytotoxicity was seen in constructs with a reduced affinity CD7 binding arm together with a high affinity CD33 binding arm in CD33+/CD7− cell lines (Table 5) and cell lines that express CD33−/CD7+ (FIGS. 7 to 9). This is the case when two different cytotoxic payloads are conjugated to the bispecific antibody. Together, this suggests that CD33+/CD7+ bispecific antibodies conjugated to a cytotoxic payload could be used to cause robust cytotoxicity in CD33+/CD7+ cells while causing reduced cytotoxicity in CD33 and CD7 single antigen positive cells, such as monocytes (CD33+/CD7−) and T cells (CD33−/CD7+).

The BVX102, BVX152, BVX162, BVX110, BVX120 and BVX130 constructs all showed promise as therapeutics, and in particular BVX130 with BVX110 and BVX 120 showing similar activity.

The forgoing embodiments are not intended to limit the scope of the protection afforded by the claims, but rather to describe examples of how the invention may be put into practice.

Sequence Listings
CD7-PP-1-WT CD7 binding arm sequence:
VH:
(SEQ ID No. 1)

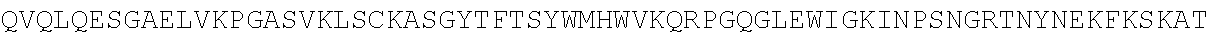

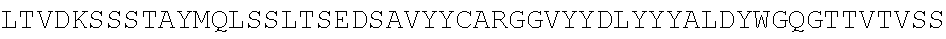

VH CDR1:
(SEQ ID No. 2)

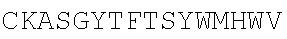

VH CDR2:
(SEQ ID No. 3)

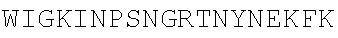

VH CDR3:
(SEQ ID No. 4)

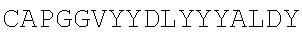

VL:
(SEQ ID No. 5)

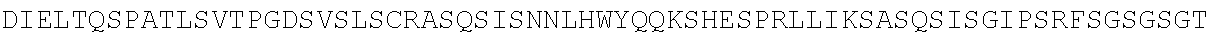

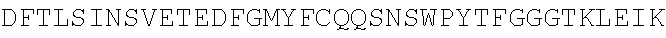

VL CDR1:
(SEQ ID No. 6)

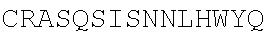

VL CDR2:
(SEQ ID No. 7)

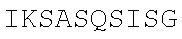

VL CDR3:
(SEQ ID No. 8)

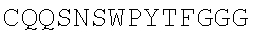

Heavy:
(SEQ ID No. 9)
QVQLQESGAELVKPGASVKLSCKASGYTFTSYWMHWVKQRPGQGLEWIGKINPSNGRTNYNEKFKSKAT
LTVDKSSSTAYMQLSSLTSEDSAVYYCARGGVYYDLYYYALDYWGQGTTVTVSSASTKGPSVFPLAPSS
KSTSGGTAALGCLVKDYFPEPVTVSWNSGALTSGVHTFPAVLQSSGLYSLSSVVTVPSSSLGTQTYCCN
VNHKPSNTKVDKKVEPKSCDKT
Light:
(SEQ ID No. 10)
DIELTQSPATLSVTPGDSVSLSCRASQSISNNLHWYQQKSHESPRLLIKSASQSISGIPSRESGSGSGT
DFTLSINSVETEDFGMYFCQQSNSWPYTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SENRGEC
CD7-PP-2 CD7 binding arm sequence:
VH:
(SEQ ID No. 11)

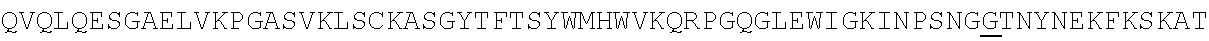

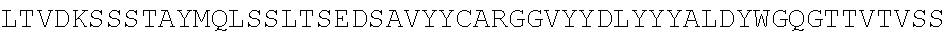

VH CDR1:
(SEQ ID No. 12)

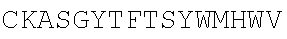

VH CDR2:
(SEQ ID No. 13)

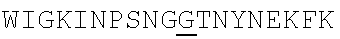

VH CDR3:
(SEQ ID No. 14)

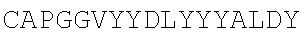

VL:
(SEQ ID No. 15)

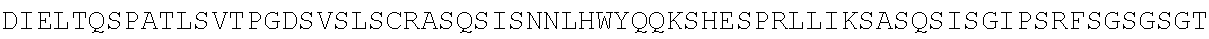

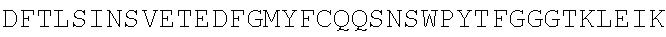

VL CDR1:
(SEQ ID No. 16)

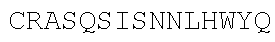

VL CDR2:
(SEQ ID No. 17)

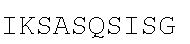

VL CDR3:
(SEQ ID No. 18)

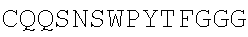

Heavy:
(SEQ ID No. 19)
QVQLQESGAELVKPGASVKLSCKASGYTFTSYWMHWVKQRPGQGLEWIGKINPSNGGTNYNEKFKSKAT
LTVDKSSSTAYMQLSSLTSEDSAVYYCARGGVYYDLYYYALDYWGQGTTVTVSSASTKGPSVEPLAPSS
KSTSGGTAALGCLVKDYFPEPVTVSWNSGALTSGVHTFPAVLOSSGLYSLSSVVTVPSSSLGTQTYCCN
VNHKPSNTKVDKKVEPKSCDKT
Light:
(SEQ ID No. 20)
DIELTQSPATLSVTPGDSVSLSCRASQSISNNLHWYQQKSHESPRLLIKSASQSISGIPSRESGSGSGT
DFTLSINSVETEDFGMYFCQQSNSWPYTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SENRGEC
CD7-PP-3 CD7 binding arm sequence:
VH:
(SEQ ID No. 21)

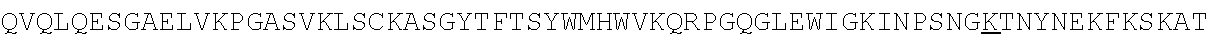

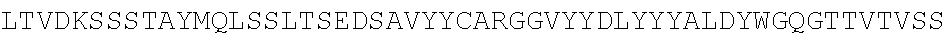

VH CDR1:
(SEQ ID No. 22)

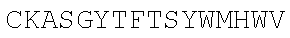

VH CDR2:
(SEQ ID No. 23)

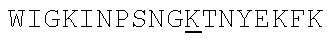

VH CDR3:
(SEQ ID No. 24)

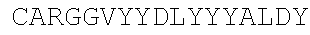

VL:
(SEQ ID No. 25)

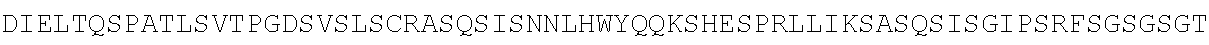

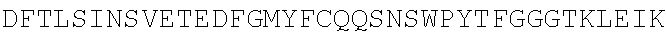

VL CDR1:
(SEQ ID No. 26)

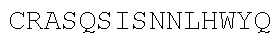

VL CDR2:
(SEQ ID No. 27)

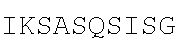

VL CDR3:
(SEQ ID No. 28)

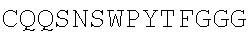

Heavy:
(SEQ ID No. 29)
QVQLQESGAELVKPGASVKLSCKASGYTFTSYWMHWVKQRPGQGLEWIGKINPSNGKTNYNEKFKSKAT
LTVDKSSSTAYMQLSSLTSEDSAVYYCARGGVYYDLYYYALDYWGQGTTVTVSSASTKGPSVFPLAPSS
KSTSGGTAALGCLVKDYFPEPVTVSWNSGALTSGVHTFPAVLQSSGLYSLSSVVTVPSSSLGTQTYCCN
VNHKPSNTKVDKKVEPKSCDKT
Light:
(SEQ ID No. 30)
DIELTQSPATLSVTPGDSVSLSCRASQSISNNLHWYQQKSHESPRLLIKSASQSISGIPSRESGSGSGT
DFTLSINSVETEDFGMYFCQQSNSWPYTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SENRGEC
CD7-PP-6 binding arm sequence:
VH:
(SEQ ID No. 31)
QVQLQESGAELVKPGASVKLSCKASGYTFTSYWMHWVKORPGQGLEWIGKINPSNGRTNYNEKFKSKAT
LTVDKSSSTAYMQLSSLTSEDSAVYYCARGGVYYALYYYALDYWGQGTTVTVSS
VH CDR1:
(SEQ ID No. 32)
CKASGYTFTSYWMHWV
VH CDR2:
(SEQ ID No. 33)
WIGKINPSNGRTNYNEKFK
VH CDR3:
(SEQ ID No. 34)
CARGGVYYALY
VL:
(SEQ ID No. 35)
DIELTQSPATLSVTPGDSVSLSCRASQSISNNLHWYQQKSHESPRLLIKSASQSISGIPSRESGSGSGT
DFTLSINSVETEDFGMYFCQQSNSWPYTFGGGTKLEIK
VL CDR1:
(SEQ ID No. 36)

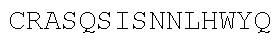

VL CDR2:
(SEQ ID No. 37)

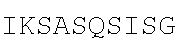

VL CDR3:
(SEQ ID No. 38)

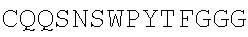

Heavy:
(SEQ ID No. 39)
QVQLQESGAELVKPGASVKLSCKASGYTFTSYWMHWVKQRPGQGLEWIGKINPSNGRTNYNEKFKSKAT
LTVDKSSSTAYMQLSSLTSEDSAVYYCARGGVYYALYYYALDYWGQGTTVTVSSASTKGPSVFPLAPSS
KSTSGGTAALGCLVKDYFPEPVTVSWNSGALTSGVHTFPAVLQSSGLYSLSSVVTVPSSSLGTQTYCCN
VNHKPSNTKVDKKVEPKSCDKT
Light:
(SEQ ID No. 40)
DIELTQSPATLSVTPGDSVSLSCRASQSISNNLHWYQQKSHESPRLLIKSASQSISGIPSRESGSGSGT
DFTLSINSVETEDFGMYFCQQSNSWPYTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SENRGEC
CD7-PP-7 CD7 binding arm sequence:
VH:
(SEQ ID No. 41)

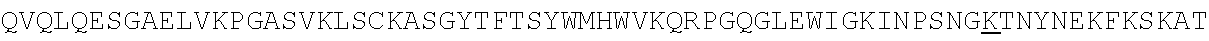

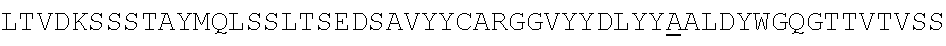

VH CDR1:
(SEQ ID No. 42)

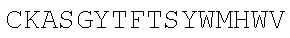

VH CDR2:
(SEQ ID No. 43)

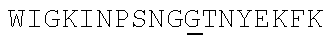

VH CDR3:
(SEQ ID No. 44)

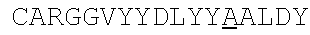

VL:
(SEQ ID No. 45)

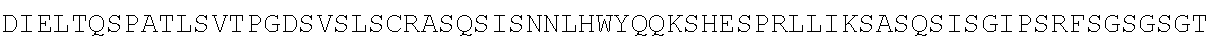

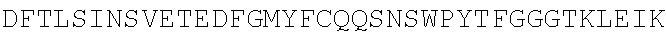

VL CDR1:
(SEQ ID No. 46)

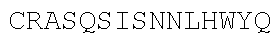

VL CDR2:
(SEQ ID No. 47)

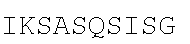

VL CDR3:
(SEQ ID No. 48)

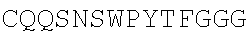

Heavy:
(SEQ ID No. 49)
QVQLQESGAELVKPGASVKLSCKASGYTFTSYWMHWVKQRPGQGLEWIGKINPSNGGTNYNEKFKSKAT
LTVDKSSSTAYMQLSSLTSEDSAVYYCARGGVYYDLYYAALDYWGQGTTVTVSSASTKGPSVFPLAPSS
KSTSGGTAALGCLVKDYFPEPVTVSWNSGALTSGVHTFPAVLQSSGLYSLSSVVTVPSSSLGTQTYCCN
VNHKPSNTKVDKKVEPKSCDKT
Light:
(SEQ ID No. 50)
DIELTQSPATLSVTPGDSVSLSCRASQSISNNLHWYQQKSHESPRLLIKSASQSISGIPSRFSGSGSGT
DFTLSINSVETEDFGMYFCQQSNSWPYTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SENRGEC
CD7-PP-8 CD7 binding arm sequence:
VH:
(SEQ ID No. 51)

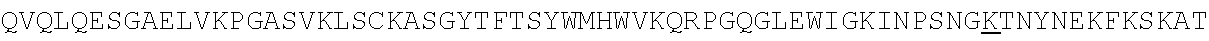

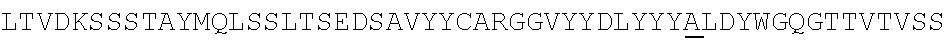

VH CDR1:
(SEQ ID No. 52)

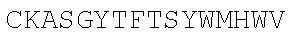

VH CDR2:
(SEQ ID No. 53)

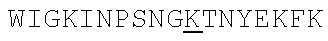

VH CDR3:
(SEQ ID No. 54)

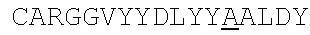

VL:
(SEQ ID No. 55)

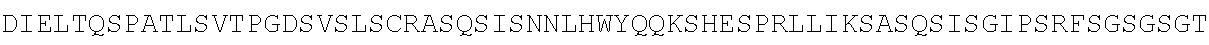

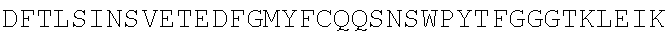

VL CDR1:
(SEQ ID No. 56)

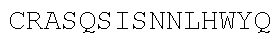

VL CDR2:
(SEQ ID No. 57)

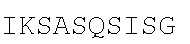

VL CDR3:
(SEQ ID No. 58)

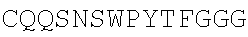

Heavy:
(SEQ ID No. 59)
QVQLQESGAELVKPGASVKLSCKASGYTFTSYWMHWVKQRPGQGLEWIGKINPSNGKTNYNEKFKSKAT
LTVDKSSSTAYMQLSSLTSEDSAVYYCARGGVYYDLYYAALDYWGQGTTVTVSSASTKGPSVFPLAPSS
KSTSGGTAALGCLVKDYFPEPVTVSWNSGALTSGVHTFPAVLQSSGLYSLSSVVTVPSSSLGTQTYCCN
VNHKPSNTKVDKKVEPKSCDKT
Light:
(SEQ ID No. 60)
DIELTQSPATLSVTPGDSVSLSCRASQSISNNLHWYQQKSHESPRLLIKSASQSISGIPSRESGSGSGT
DFTLSINSVETEDFGMYFCQQSNSWPYTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SENRGEC
CD7-PP-12 CD7 binding arm sequence:
VH:
(SEQ ID No. 61)

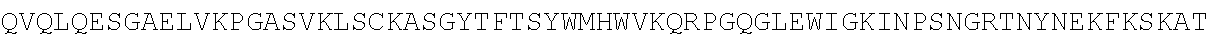

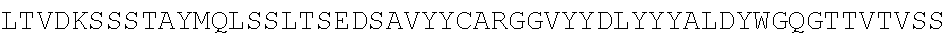

VH CDR1:
(SEQ ID No. 62)

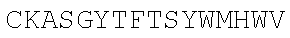

VH CDR2:
(SEQ ID No. 63)

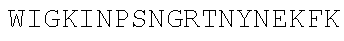

VH CDR3:
(SEQ ID No. 64)

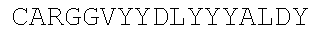

VL:
(SEQ ID No. 65)

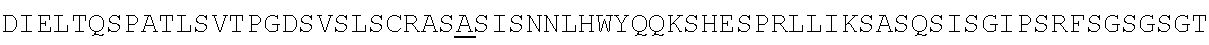

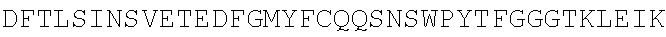

VL CDR1:
(SEQ ID No. 66)

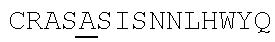

VL CDR2:
(SEQ ID No. 67)

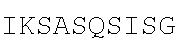

VL CDR3:
(SEQ ID No. 68)

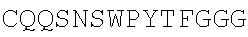

Heavy:
(SEQ ID No. 69)
QVQLQESGAELVKPGASVKLSCKASGYTFTSYWMHWVKQRPGQGLEWIGKINPSNGRTNYNEKFKSKAT
LTVDKSSSTAYMQLSSLTSEDSAVYYCARGGVYYDLYYYALDYWGQGTTVTVSSASTKGPSVFPLAPSS
KSTSGGTAALGCLVKDYFPEPVTVSWNSGALTSGVHTFPAVLQSSGLYSLSSVVTVPSSSLGTQTYCCN
VNHKPSNTKVDKKVEPKSCDKT
Light:
(SEQ ID No. 70)
DIELTQSPATLSVTPGDSVSLSCRASASISNNLHWYQQKSHESPRLLIKSASQSISGIPSRESGSGSGT
DETLSINSVETEDFGMYFCQQSNSWPYTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SENRGEC
CD7-PP-13 CD7 binding arm sequence:
VH:
(SEQ ID No. 71)

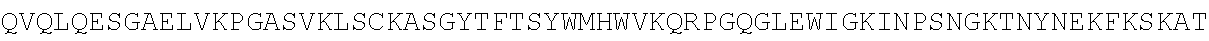

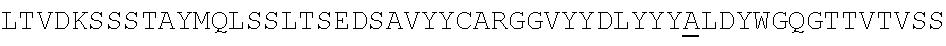

VH CDR 1:
(SEQ ID No. 72)

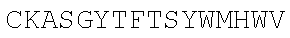

VH CDR 2:
(SEQ ID No. 73)

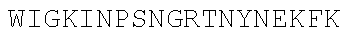

VH CDR 3:
(SEQ ID No. 74)

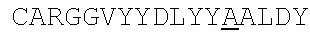

VL:
(SEQ ID No. 75)

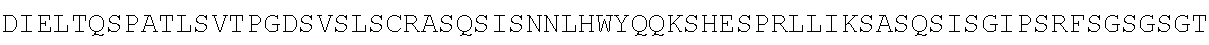

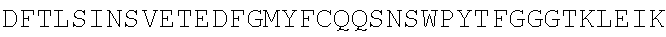

VL CDR1:
(SEQ ID No. 76)

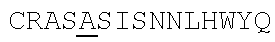

VL CDR2:
(SEQ ID No. 77)

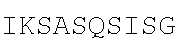

VL CDR3:
(SEQ ID No. 78)

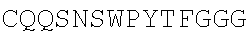

Heavy:
(SEQ ID No. 79)
QVQLQESGAELVKPGASVKLSCKASGYTFTSYWMHWVKQRPGQGLEWIGKINPSNGRTNYNEKFKSKAT
LTVDKSSSTAYMQLSSLTSEDSAVYYCARGGVYYDLYYAALDYWGQGTTVTVSSASTKGPSVFPLAPSS
KSTSGGTAALGCLVKDYFPEPVTVSWNSGALTSGVHTFPAVLQSSGLYSLSSVVTVPSSSLGTQTYCCN
VNHKPSNTKVDKKVEPKSCDKT
Light:
(SEQ ID No. 80)
DIELTQSPATLSVTPGDSVSLSCRASQSISNNLHWYQQKSHESPRLLIKSASQSISGIPSRESGSGSGT
DFTLSINSVETEDFGMYFCQQSNSWPYTFGGGTKLEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SENRGEC
CD33-PP-1-WT binding arm sequence:
VH:
(SEQ ID No. 81)
EVQLVQSGAEVKKPGSSVKVSCKASGYTITDSNIHWVRQAPGQSLEWIGYTYPYNGGTDYNQKEKNRAT
LTVDNPTNTAYMELSSLRSEDTAFYYCVNGNPWLAYWGQGTLVTVSS
VH CDR 1:
(SEQ ID No. 82)

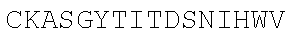

VH CDR 2:
(SEQ ID No. 83)

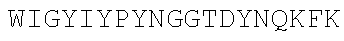

VH CDR 3:
(SEQ ID No. 84)

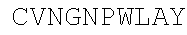

VL:
(SEQ ID No. 85)
DIQLTQSPSTLSASVGDRVTIICRASESLDNYGIRFLTWFQQKPGKAPKLLMYAASNQGSGVPSRFSGS
GSGTEFTLTISSLQPDDFATYYCQQTKEVPWSFGQGTKLEIK
VL CDR 1:
(SEQ ID No. 86)

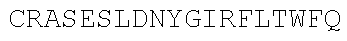

VL CDR 2:
(SEQ ID No. 87)

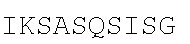

VL CDR 3:
(SEQ ID No. 88)

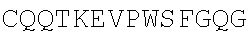

CD33-PP-4 binding arm sequence:
VH:
(SEQ ID No. 89)
EVQLVQSGAEVKKPGSSVKVSCKASGYTITDSNIHWVRQAPGQSLEWIGYTYPYNGGTDYNQKEKNRAT
LTVDNPTNTAYMELSSLRSEDTAFYYCVNGNPYLAYWGQGTLVTVSS
VH CDR 1:
(SEQ ID No. 90)

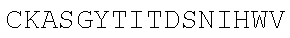

VH CDR 2:
(SEQ ID No. 91)

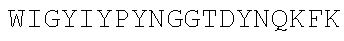

VH CDR 3:
(SEQ ID No. 92)

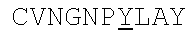

VL:
(SEQ ID No. 93)
DIQLTQSPSTLSASVGDRVTIICRASESLDNYGIRFLTWFQQKPGKAPKLLMYAASNQGSGVPSRESGS
GSGTEFTLTISSLQPDDFATYYCQQTKEVPWSFGQGTKLEIK
VL CDR 1:
(SEQ ID No. 94)

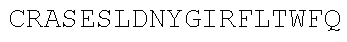

VL CDR 2:
(SEQ ID No. 95)

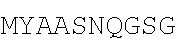

VL CDR 3:
(SEQ ID No. 96)

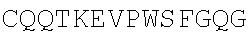

CD33-PP-7 binding arm sequence:
VH:
(SEQ ID No. 97)
EVQLVQSGAEVKKPGSSVKVSCKASGYTITDSNIHWVRQAPGQSLEWIGYIYPYNGGTDYNQKFKNRAT
LTVDNPTNTAYMELSSLRSEDTAFYYCVNGNPYLAAWGQGTLVTVSS
VH CDR 1:
(SEQ ID No. 98)

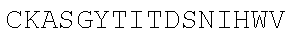

VH CDR 2:
(SEQ ID No. 99)

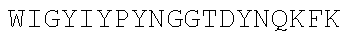

VH CDR 3:
(SEQ ID No. 100)

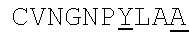

VL:
(SEQ ID No. 101)
DIQLTQSPSTLSASVGDRVTIICRASESLDNYGIRFLTWFQQKPGKAPKLLMYAASNQGSGVPSRESGS
GSGTEFTLTISSLQPDDFATYYCQQTKEVPWSFGQGTKLEIK
VL CDR 1:
(SEQ ID No. 102)

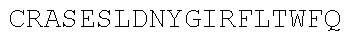

VL CDR 2:
(SEQ ID No. 103)

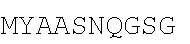

VL CDR 3:
(SEQ ID No. 104)

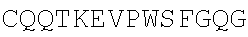

Optional Signal Sequence Upstream of VH and VL Domains
(SEQ ID No. 105)
METDTLLLWVLLLWVPGST

1. A bispecific antibody or antigen binding fragments thereof binding to CD33 and CD7, wherein the bispecific antibody or antigen binding fragments comprises:
a first binding region binding to human CD33 which has a K_{D} binding affinity to CD33 of between about 1.36×10⁻⁹ and about 9.23×10⁻⁸; and
a second binding region binding to human CD7 which has a K_{D} binding affinity to CD7 of between about 3.56×10⁻⁹ and about 2.29 ×10⁻⁷.

2. The antibody or antigen binding fragments thereof according to claim 1, wherein the first binding region binding to human CD33 has a K_{D} binding affinity to CD33 of about 1.36×10⁻⁹ and the second binding region binding to human CD7 which has a K_{D} binding affinity to CD7 of about 4.52 ×10⁻⁸.

3. The antibody or antigen binding fragments thereof according to claim 1, wherein the first binding region has a reduced K_{D} binding affinity for CD33 compared to a wild type binding region that is able to bind to CD33.

4. The antibody or antigen binding fragments thereof according to claim 3, wherein the first binding region has a reduced K_{D} binding affinity for CD33 of up to about 70 fold.

5. The antibody or antigen binding fragments thereof according to claim 4, wherein the first binding region has a reduced K_{D} binding affinity for CD33 of up to about 68 fold.

6. The antibody or antigen binding fragments thereof according to claim 1, wherein the first binding region does not has a reduced K_{D} binding affinity for CD33 compared to a wild type binding region.

7. The antibody or antigen binding fragments thereof according to claims 1 to 5, wherein the second binding region has a reduced binding affinity for CD7 compared to a wild type binding region that is able to bind to CD7.

8. The antibody or antigen binding fragments thereof according to claim 7, wherein the second binding region has a reduced K_{D} binding affinity for CD7 of up to about 90 fold.

9. The antibody or antigen binding fragments thereof according to claim 8, wherein the second binding region has a reduced K_{D} binding affinity for CD7 of up to about 87 fold.

10. The antibody or antigen binding fragments thereof according to claim 7, wherein the second binding region has a reduced K_{D} binding affinity for CD7 in the range of about 9 to about 90 fold.

11. The antibody or antigen binding fragments thereof according to claim 10, wherein the second binding region has a reduced K_{D} binding affinity for CD7 in the range of about 9 to about 87 fold.

12. The antibody or antigen binding fragments thereof according to claim 1, wherein both the first and/or second binding regions have a reduced binding affinity for their respective binding targets compared to respective wildtype binding regions ability to bind to the respective binding targets.

13. The antibody or antigen binding fragments thereof according to claim 12, wherein the first binding region has substantially the same K_{D} binding affinity to CD33 when compared to a wild type binding region that is able to bind to CD33 and wherein the second binding region of has a reduced K_{D} binding affinity for CD7 when compared to a wild type binding region that is able to bind to CD7.

14. The antibody or antigen binding fragments thereof according to claims 12 and 13 wherein the second binding region has a K_{D} binding affinity for CD7 that is between about 9 and about 87 fold lower than that of a wild type binding region that is able to bind to CD7.

15. The antibody or antigen binding fragments thereof according to any preceding claim wherein: a) the first binding region has a K_{D} binding affinity for CD33 that is selected from one of the following: about 1.36×10⁻⁹ or about 9.23×10⁻⁸; and/or b) the second binding region binding to human CD7 has a K_{D} binding affinity to CD7 selected from one of the following: about 3.56×10⁻⁹, about 7.22×10⁻⁸, about 7.57×10⁻⁸, about 4.52×10⁻⁸, about 3.25×10^{-8 ,} about 5.41×10⁻⁷ or about 2.29×10⁻⁷.

16. The antibody or antigen binding fragments thereof according to claim 1, having a fold selectivity of CD7⁺/CD33⁺ vs CD7⁺/CD33⁻ in the range of about 2 to about 20 and a fold selectivity of CD7⁺/CD33⁺ vs CD7⁻/CD33⁺ in the range of about 20 to about 70.

17. The antibody or antigen binding fragments thereof according to claim 16, having a fold selectivity of CD7⁺/CD33⁺ vs CD7⁺/CD33⁻ in the range of about 5 to about 15 and a fold selectivity of CD7⁺/CD33⁺ vs CD7⁻/CD33⁺ in the range of about 20 to about 60.

18. The antibody or antigen binding fragments thereof according to claim 17, having a fold selectivity of CD7⁺/CD33⁺ vs CD7⁺/CD33⁻ in the range of about 5 to about 15 and a fold selectivity of CD7⁺/CD33⁺ vs CD7⁻/CD33⁺ in the range of about 25 to about 55.

19. The antibody or antigen binding fragments thereof according to claim 18, having a fold selectivity of:
a) CD7⁺/CD33⁺ vs CD7⁺/CD33⁻ of about 14 and a fold selectivity of CD7⁺/CD33⁺ vs CD7⁻/CD33⁺ of about 48;
b) CD7⁺/CD33⁺ vs CD7⁺/CD33⁻ of about 6 and a fold selectivity of CD7⁺/CD33⁺ vs CD7⁻/CD33⁺ of about 48; or
c) CD7⁺/CD33⁺ vs CD7⁺/CD33⁻ of about 7 and a fold selectivity of CD7⁺/CD33⁺ vs CD7⁻/CD33⁺ of about 27.

20. The antibody or antigen binding fragments thereof according to claim 19, having a fold selectivity of:
a) CD7⁺/CD33⁺ vs CD7⁺/CD33⁻ of about 13.6 and a fold selectivity of CD7⁺/CD33⁺ vs CD7⁻/CD33⁺ of about 48.3;
b) CD7⁺/CD33⁺ vs CD7⁺/CD33⁻ of about 6.5 and a fold selectivity of CD7⁺/CD33⁺ vs CD7⁻/CD33⁺ of about 27.4; or
c) CD7⁺/CD33⁺ vs CD7⁺/CD33⁻ of about 6.1 and a fold selectivity of CD7⁺/CD33⁺ vs CD7⁻/CD33⁺ of about 47.5.

21. The antibody or antigen binding fragments thereof according to any one of claims 1 to 20 for use in the treatment of a CD7+CD33+ hematological malignancy.

22. The antibody or antigen binding fragments thereof for use according to claim 21, wherein said antibody or antigen binding fragments thereof is capable of inducing CD33 and/or CD7 receptor mediated internalization into a CD33+ and/or CD7+ cell.

23. The antibody or antigen binding fragments thereof for use according to claim 21 or 22, wherein the antibody or antigen binding fragments thereof bispecifically binds CD33 and CD7 and wherein the CD33+ and CD7+ cell is an AML cell.

24. The antibody or antigen binding fragments thereof for use according to any one of claims 21 to 23, wherein the antibody or antigen binding fragments thereof is capable of mediating antibody dependent cellular cytotoxicity.

25. The antibody or antigen binding fragments thereof for use according to any one of claims 21 to 23, wherein the antibody or antigen binding fragments thereof is attached to, or formed with an immune effector cell.

26. The antibody or antigen binding fragments thereof for use according to claim 25, wherein the immune effector cell comprises a T cell and/or a NK cell.

27. The antibody or antigen binding fragments thereof for use according to claim 26, wherein the cell inhibiting cell is a T cell.

28. The antibody or antigen binding fragments thereof for use according to claim 27, wherein the immune effector cell is a bispecific anti-CD33 anti-CD7 CAR-T.

29. The antibody or antigen binding fragments thereof for use according to claim 27, wherein the T cell comprises a CD33+ T cell, a CD7+ T cell, or a combination thereof.

30. The antibody or antigen binding fragments thereof for use according to any one of claims 25 to 26, wherein the antibody or antigen fragments thereof comprises: i) a cell killing portion; ii) a CD7 binding portion and iii) a CD33 binding portion.

31. The antibody or antigen binding fragments thereof for use according to claim 30, wherein said CD33 binding portion comprises an antigen binding fragments of an antibody.

32. The antibody or antigen binding fragments thereof for use according to claim 30 or claim 31, wherein said CD7 binding portion comprises an antigen binding fragments of an antibody.

33. The antibody or antigen binding fragments thereof for use according to any one of claims 30 to 32, wherein said cell killing portion is a cytotoxin.

34. The antibody or antigen binding fragments thereof for use according to claim 33, wherein said cytotoxin is selected from: i) a peptide toxin or ii) a chemical toxin.

35. The antibody or antigen binding fragments thereof for use according to any one of claims 30 to 32, further comprises a linking portion linking the cell kill portion with the CD7 binding portion and/or the CD33 binding portion.

36. The antibody or antigen binding fragments thereof for use according to any one of claims 30 to 34, in the format of an antibody drug conjugate.
